(19) United States
(12) Patent Application Publication
(10) Pub. No.: US 2024/0229158 A1
(43) Pub. Date: Jul. 11, 2024

(54) DNA METHYLATION BIOMARKERS FOR HEPATOCELLULAR CARCINOMA

(71) Applicant: OPHIOMICS - INVESTIGAÇÃO E DESENVOLVIMENTO EM BIOTECNOLOGIA, Lisboa (PT)

(72) Inventors: José PEREIRA LEAL, Lisboa (PT); Joana CARDOSO VAZ, Lisboa (PT); Emanuel José VIEIRA GONÇALVES, Lisboa (PT); Maria Ana GONÇALVES REIS, Lisboa (PT)

(73) Assignee: OPHIOMICS - INVESTIGAÇÃO E DESENVOLVIMENTO EM BIOTECNOLOGIA, Lisboa (PT)

(21) Appl. No.: 18/562,839

(22) PCT Filed: May 23, 2022
(86) PCT No.: PCT/EP2022/063902

§ 371 Date: Nov. 21, 2023

(30) Foreign Application Priority Data
May 21, 2021 (EP) 21175425.4

(51) Int. Cl. C12Q 1/6886; G16B 20/20; G16B 40/20

(52) U.S. Cl. CPC C12Q 1/6886; G16B 20/20; G16B 40/20; C12Q 2600/154

(57) ABSTRACT

The invention provides a robust method to detect cancer in DNA extracted from an exploratory tissue biopsy or plasma sample obtained from a patient, comprising measuring a level of DNA methylation at a plurality of defined differentially methylated regions of the genome comprising multiple CpG sites.

The present invention relates to an advantageous method for detecting low concentrations of cancer-derived DNA in patient samples by determining the DNA methylation signature at a plurality of genetic loci.

The present invention claims the benefit of the priority of European patent application EP21175425, filed 21 May 2021, which is full incorporated herein by reference.

### BACKGROUND OF THE INVENTION

Current HCC diagnostic guidelines require the usage of invasive procedures, such as tissue biopsies, followed by histological and/or contrast-enhanced imaging. These time-consuming procedures contribute to HCC being most often detected at an advanced stage, where 40% of the cases are multinodular or metastatic, and leaving 72% of the cases without any treatment options (Llovet et al. 2021 Nat. Rev. Dis. Primers 7:6). Screening and surveillance programmes are therefore vital to detect and diagnose HCC in early stages and provide patients with a larger time window for therapeutic options which may extend life expectancy.

Liquid biopsies (LBs) from body fluids, for example plasma and urine, contain circulating molecular biomarkers of HCC have potential as non-invasive and inexpensive alternatives for early diagnosis assays. High levels of alpha-fetoprotein (AFP) in such samples can identify HCC with almost perfect specificity, but sensitivity (recall) rates are frequently low, at less than 45%, while lower thresholds of AFP (20 ng/ml) balance between specificity and sensitivity with both ranging around 79%. Of note, in patients with chronic liver disease, in particular HCV-related cirrhosis, the precision of AFP as a biomarker is significantly reduced and insufficient for robust diagnosis (Bialecki et al. 2005 HPB 7:26). LBs also contain cell-free DNA (cfDNA) material derived from cells throughout the body, including circulating tumour DNA (ctDNA). Measurement of genetic markers in ctDNA, such as mutations and methylation, can be used as a diagnostic and therapeutic tool.

Several studies have identified DNA methylation biomarkers for HCC (Liu et al. 2020 Ann. Oncol. 31, 745; Bonder et al. 2014 BMC Genomics 15, 860; Wang et al. 2019 Hepatology 70, 51; Chang et al. 2018 Genome Med. 10, 42; Shui et al. 2020 Front. Genet. 11, 906), but were limited to either tissue samples only, focused on the identification of small sets of single CpG sites, and/or compared to healthy liver tissue samples and thus were unable to differentiate HCC from chronic liver disease, for example cirrhosis, as well as being vulnerable to probe failures or the presence of a mutation in a patient sample precluding a diagnostic result.

Based on the above-mentioned state of the art, the objective of the present invention is to provide means and methods to accurately detect low concentrations of tumour-derived DNA in a patient sample, particularly to detect the presence of HCC-derived DNA in a cell free sample such as plasma.

This objective is attained by the subject-matter of the independent claims of the present specification, with further advantageous embodiments described in the dependent claims, examples, figures and general description of this specification.

### SUMMARY OF THE INVENTION

The invention relates to a method to detect a DNA methylation signal specific to cancer cells in patient samples, even when the cancer cell DNA is present at very low concentrations, for example, cell-free tumour DNA present in plasma samples obtained from a patient suspected of having cancer in a certain organ, particularly a patient suspected of having hepatocellular carcinoma.

The method comprises measuring a level of methylation at a plurality of differentially methylated regions (DMR) of the genome, to obtain a value for each DMR which reflects the methylation status one or more redundant CpG sites which share a distinct cancer-specific methylation signature. The method further comprises evaluating the statistical significance of the plurality of DMR methylation values, in order to assign the patient a high, or a low probability of having cancer.

The method according to the invention advantageously incorporates predictive information from multiple redundant methylation measurements, so that in the event of the failure of one or several individual components of the method, for example, a failure to obtain a single CpG measurement due to the presence of a single nucleotide polymorphism in the patient DNA, or a technical failure of one or more assay probes, a patient may still be accurately assigned a probability of having cancer based on other measurements that were successfully determined.

The DMRs are delimited in such a way that the DNA methylation of a single CpG sites within the DMR, provides equivalent cancer predictive value to the average 2 or more, or all the CpG sites within a DMR. A second layer of redundancy which enhances the sensitivity of this diagnostic method is introduced by flexible combination of the predictive value of 2 to 38, particularly 8 to 38, more particularly 10 to 20 of the DMR specified in Table 1 into a predictive risk score, in order to create a method which will accurately assign patients probability of having cancer based on the DNA methylation signature of an ex vivo sample.

Particular embodiments of the invention relate to inputting the DMR methylation levels into a cancer-predicting classification algorithm to obtain a risk score, then assigning a patient a probability that the patient has cancer, and optionally comparing the risk score to a threshold.

Particular embodiments of the invention relate to the use of the method according to the invention above to analyse a plasma sample, or a liver biopsy sample, in order to determine whether a patient has hepatocellular carcinoma.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One such example for comparison of nucleic acid sequences is the BLASTN algorithm that uses the default settings: Expect threshold: 10; Word size: 28; Max matches in a query range: 0; Match/Mismatch Scores: 1 .- 2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively. Reference to identical sequences without specification of a percentage value implies 100% identical sequences (i.e. the same sequence).

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The term probe in the context of the specification relates to a molecular probe, particularly a nucleic acid probe capable of selectively hybridizing to a specific region comprising a single target CpG dinucleotide. Such hybridizing nucleic acid sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and on the backbone chemistry.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues. In certain embodiments, a hybridizing sequence according to the invention comprises 8, 9, 10, 11, 12, 13, 14, 15, 16, 17, 18, 19, 20, 21, 22, 23, 24, 25, 26, 27, 28, 29 or 30 nucleotides. In certain embodiments, the hybridizing sequence is at least 80% identical, more preferred 85%, 90%, 92%, 94%, 95%, 96%, 97%, 98% or 99% identical to the reverse complimentary sequence of surrounding a CpG site listed in Table 1. In certain embodiments, the hybridizing sequence comprises deoxynucleotides, phosphothioate deoxynucleotides, LNA and/or PNA nucleotides or mixtures thereof.

The terms CPG site, CpG locus or CpG residue, sometimes abbreviated to cg in CpG site nomenclature, in the context of the present specification relate to CpG DNA dinucleotides which may be either methylated or unmethylated as described above. A CpG dinucleotide is a position in the genome where a cytosine nucleotide is joined by a phosphodiester bond to a guanine nucleotide (in the 5′ to 3′ direction). In humans, DNA methylation occurs at the 5′ position of the pyrimidine ring of cytosine residues. The CpG sites specified herein in Table 1 refer to those CpG sites where differential methylation may be accurately detected in both liquid, cell-free samples, such as plasma, or liver tissue samples, in a patient suffering from cancer, particularly hepatocellular carcinoma patients, compared to samples from healthy controls, or samples from patients with non-cancer disease.

The term DNA methylation level, DNA methylation, or methylation level in the context of the present specification refers to the presence, or absence of methylated CpG dinucleotide motifs at a specified genetic locus, either at one CpG site, or at one or more CpG sites within a differentially methylated region (see below). In terms of the data presented in the examples, DNA methylation of a CpG site is represented using beta methylation values, normalised measurements obtained from the fluorescence signal intensity generated by probes binding to either bisulphite-modified unmethylated, or a methylated alleles at a certain target CpG site in the genome in a methylation microarray. Beta methylation as used herein standardises raw measurements related to the presence of methylated and unmethylated motifs within a limited range, from 0, indicating hypomethylation of a particular target CpG dinucleotide site, and 1, indicating hypermethylation of the site, expressed relative to the total amount of DNA comprising the target CpG present in the sample, and offset by a fixed value specific to the mode of measurement and recommended by the manufacturer.

The term differentially methylated region, or DMR refers to genomic regions, sometimes referred to herein as CpG clusters in which a differential methylation status is present in two groups. In the context of the present specification the 38 DMR of particular interest according to the invention due to their different methylation signature cancer and non-cancer sample are listed in Table 1 alongside their position in human reference genome 38. DMR 1 through 38 contain at least 3 CpG sites, and no two consecutive CpG sites are more than 500 base pairs apart. The methylation of a DMR refers to the level of methylation measured at one of said CpG sites, or the average, or median of a level methylation of more than one of said CpG sites.

The term cancer in the context of the present specification refers to a malignant neoplastic disease in which tumour cells proliferate uncontrollably, and encompasses both primary tumours and metastatic disease. Importantly, tumour cells are often characterised by aberrant DNA methylation compared to healthy controls, or other inflammatory diseases. Differential DNA methylation specific to cancer can be detected in tumour biopsy samples containing large amounts of tumour DNA, but also in samples containing very low concentrations of cell free DNA, such as urine, plasma, serum or blood by means of a sufficiently sensitive diagnostic assay. The term cancer according to the invention encompasses solid tumours, such as lung, liver, or colon cancer, and blood-cell derived cancers such as lymphoma or leukaemia. The term cancer according to the invention encompasses both a primary cancer, and the recurrence of a cancer disease.

The term patient in the context of the present specification encompasses a subject suspected of having cancer, or a patient previously diagnosed with cancer and undergoing monitoring for disease relapse.

The term liver cancer according to the invention refers to cancers originating from liver cells, such as hepatocellular carcinoma (HCC), derived from hepatocytes, and intrahepatic cholangiocarcinoma. A patient with HCC encompasses those which also suffer from a comorbidity affecting the liver, such as hepatitis C infection, or cirrhosis.

The term chronic liver disease in the context of the specific invention, refers to non-cancer disease characterised by inflammation of the liver, including, but not limited to, infections with a virus such as Hepatitis A or C, patients with alpha-1 antitrypsin deficiency, inflammation associated with obesity, and cirrhosis. The control samples used in comparisons with cancer sample to identify predictive DMR according to the examples make use of such chronic liver disease samples in order to identify methylation signatures which differentiate samples comprising cancer cells from samples characterised by non-cancer inflammation which effects liver function. Samples obtained from patients diagnosed with chronic liver disease according to the invention are of use to train predictive algorithms according to the invention.

The term cirrhosis, refers to chronic liver disease marked by liver cell death, inflammation, and fibrosis. Cirrhosis is often a precursor to HCC. Cirrhosis may arise due to genetic mutations, viral infection, exposure to toxins or alcohol consumption.

### DETAILED DESCRIPTION OF THE INVENTION

A first aspect of the invention is a method to determine whether a patient has cancer comprising the following steps:

A measurement step, where a level of DNA methylation level is determined for a plurality of differentially methylation regions (DMR) in an ex-vivo sample obtained from the patient. The plurality of DMR according to the invention comprises, or essential consists of any two, or more of the DMR specified in Table 1, each DMR comprising 3 or more CpG sites characterized by differential methylation in cancer and non-cancer samples.

In some embodiments, the DNA methylation level of any DMR as specified above according to the invention may be the DNA methylation level determined for a single of the CpG sites listed within that DMR according to Table 1. For example, the methylation level of DMR1 may be the methylation level measured at one of cg144855744, cg20547777, or cg16009311.

Other embodiments relate to the use of the average methylation level of 2 or more CpG sites comprised within any DMR to provide a level of methylation for a DMR. For example, the methylation level of DMR1 maybe be the average of the individual levels of DNA methylation determined at
cg144855744 and cg20547777,
cg144855744 and cg16009311,
cg20547777 and cg16009311, or
cg144855744, cg20547777, and cg16009311.

The number of CpG sites at which a DNA methylation level is measured within each DMR is not particularly limited to the invention, as each provides equivalent cancer-predicative information, as demonstrated in FIG. 7 of the examples.

The next step of the method is an evaluation step, where the combined statistical significance of the plurality of DMR methylation levels determined in the measurement step is assessed. Assessing the statistical significance of the plurality of DMR methylation levels may include, for example, comparing the methylation values to control samples previously determined to contain, or not contain DNA derived from cancer cells, or to a threshold value representative of the methylation levels of said control samples, by assessing whether each DMR is characterized by hypo- or hypermethylation in comparison to said control or threshold value, or by combining the plurality of DNA methylation values obtained for each DMR into an algorithm which delivers a single numerical value reflecting the global DMR methylation signature of the sample.

Next, in an assignment step, the patient is assigned either a high probability of having cancer, or a low probability of having cancer based on the combined statistical significance of the plurality of DMR methylation levels obtained in the evaluation step.

In an optional further step, a patient assigned a high probability of having cancer can be treated with an appropriate antineoplastic therapy or particular cancer-specific treatment regimen, such as with one or more chemotherapeutic agents or checkpoint inhibitors, as described herein. Alternatively, a patient assigned a low probability of having cancer will require no treatment, or additional testing for cancer 2, 4, 6, 8, 10, 12 or more months following the initial low probability assignment.

The number of DMR for which a methylation level is obtained may vary according to various embodiments of the invention, and according to the methodology with which the methylation level is obtained, or the accuracy or sensitivity desired in the diagnostic assay.

Some embodiments relate to a method wherein a DMR methylation level is determined for between 2 to 38 of the DMR specified in Table 1, as even incorporating the DNA methylation level of 2 DMR in a risk score is demonstrated to achieve more than 80% sensitivity and more than 90% precision classifying patient samples with and without cancer (Table 7).

Other embodiments relate to a method wherein a DMR methylation level is determined for between 8 to 38 of the DMR specified in Table 1, as using the DNA methylation level of 8 DMR in a risk score classifies patient samples according to presence of HCC in the patient with a sensitivity rate of over 90%.

Particular embodiments relate to a method wherein a DMR methylation level is determined for about 20 of the DMR listed in Table 1, demonstrated in Table 2 of the examples to achieve a sensitivity rate of over 95% when used in a predictive additive linear algorithm to obtain a risk score which classifies patients according to the presence or absence of HCC-derived DNA in a patient sample.

The method according to the invention may be used to detect the presence of cancer cells in a patient sample. Some embodiments relate to the use of the diagnostic method according to the invention to identify a DNA methylation signature indicative of lung, colon, breast, or liver cancer.

Particular embodiments of the invention relate to the use of the method as specified above to detect a DNA methylation signature in DNA extracted from a patient sample in order to determine whether the patient does, or does not have hepatocellular carcinoma.

As the method according to the invention is both sensitive and robust, the method is expected to be broadly applicable to many different types of ex vivo patient samples.

Particular embodiments relate to use of DNA extracted from an exploratory biopsy of a tissue in which cancer is suspected to be present.

Other embodiments relate to the use of DNA extracted from a liquid tissue sample, such as blood, or even a cell-free sample such as plasma or serum.

Particular embodiments relate to use of DNA extracted from plasma obtained from a patient suspected of having a cancer originating from a solid organ, for example HCC.

Some embodiments of the invention relate to assigning a patient a high probability of having cancer if the methylation level determined for DMR2, DMR4, DMR5, DMR9, DMR10, DMR14, DMR15, DMR16, DMR18, DMR23, DMR24, DMR28, DMR29, DMR35, and/or DMR37 indicates the region is hypermethylated, and/or if the methylation level determined for DMR1, DMR3, DMR6, DMR7, DMR8, DMR11, DMR12, DMR13, DMR17, DMR19, DMR20, DMR21, DMR22, DMR25, DMR26, DMR27, DMR30, DMR31, DMR32, DMR33, DMR34, DMR36, and/or DMR38 indicates the region is hypomethylated.

Hypermethylation or hypomethylation according to this embodiment of the invention may be ascertained in the evaluation step in reference to an average, or median methylation level of said DMR as determined in a plurality of control samples previously determined to be free of cancer cells, particularly within 2, or more particularly 1 standard deviation from said average.

In other embodiments of the evaluation step, the plurality of DNA methylation levels are submitted to a predictive, classification algorithm which classifies the sample according to the probability that the sample contains DNA derived from a cancer cell, to obtain a risk score.

Particular embodiments relate to use of an additive linear score as a classification algorithm according to the invention.

Particularly embodiments relate to submitting the plurality of DNA methylation levels obtained in the measurement step into an additive linear score by
multiplying each of the plurality of DMR methylation levels by an individual weighting value calculated according to a relative predictive power observed for any one DMR, to obtain a plurality of weighted DMR methylation values, and
calculating the sum of the plurality weighted DMR methylation values to obtain a risk score.

The relative predictive power of any one DMR, is a function of the amount, and variability of DNA methylation observed between the plurality of HCC and non HCC patient samples Test and Validation cohorts used in the examples. The Top 38, 20, 10, 8, 5, 3 and 2 predictive DMR for HCC are listed in Table 1 through 7 of the examples.

Some embodiments of the measurement step relate to determination of the methylation level at a plurality of DMR comprising the top predictive region DMR1.

Other embodiments of the measurement step relate to determination of the methylation level at a plurality of DMR comprising or consisting of the top 2 predictive regions, DMR1 and DMR4.

Other embodiments of the measurement step relate to determination of the methylation level at a plurality of DMR comprising or consisting of the top 3 predictive regions, DMR1, DMR4, and DMR28,
Other embodiments of the measurement step relate determination of the methylation level at a plurality of DMR comprising or consisting of the top 5 predictive regions, DMR1, DMR4, DMR28, DMR35, and DMR36,

Particular embodiments of the measurement step to determination of the methylation level at a plurality of DMR comprising or consisting of the top 8 predictive regions, DMR1, DMR4, DMR6, DMR7, DMR31, DMR35, DMR28 and DMR23,

Particular embodiments of the measurement step relate to determination of the methylation level at a plurality of DMR comprising or consisting of the top 10 predictive regions, DMR1, DMR4, DM27, DMR6, DMR2, DMR16, DMR31, DMR35, DMR28 and DMR23.

The multi-cohort meta-analysis presented in the examples demonstrate a predictive risk score incorporating information derived from the size and variability hyper- or hypo- DNA methylation at between 2 to 38 DMR in two groups of samples which either did, or did not contain cancer-derived cells. When compared to a threshold value depending on the number of DMR values incorporated, said predictive risk score can robustly identify whether a cancer cell, particularly an HCC cell-derived DNA methylation signature is present or not in a patient sample, whether that patient sample is a liver tissue sample, or a serum sample.

Some embodiments of the assignment step relate specified above relate to a process of a comparing of a risk score as specified above to a threshold value which accurately discriminates cancer and non-cancer samples. In some embodiments, a risk score obtained by inputting a plurality of DMR methylation values into a predicative algorithm as specified above, which is equal or above (≥) a threshold indicates that the patient has a high probability of having cancer. Conversely, a risk score below (<) the threshold indicates that the patient has a low probability of having cancer.

Particular embodiments of the invention relating to the use of a predictive algorithm in the evaluation step, obtained by training a classification model. A classification model uses an input of training values to develop an algorithm which can catagorise new values. Suitable classification models according to the invention include, but are not limited to, a logistic classification model, or an elastic net classification model, particularly a ridge regression classification model. The data demonstrates in the cohort studied in the examples demonstrates obtaining suitable coefficients, or individual weighting values to apply to the DMR methylation value as part of an additive linear score using a ridge regression classification model with a regularisation parameter of 1.

In certain embodiments relating to training a classification model with a plurality of samples obtained from patients previously determined to have, or not have cancer, the cohort of training samples according to this embodiment of invention comprises roughly equal proportions of
cell free samples, such as plasma samples previously determined to contain cancer-derived DNA,
tissue biopsies previously determined to contain cancer-derived DNA,
cell free samples, such as plasma samples from healthy subjects and/or patients with other diseases, such as chronic liver disease, or sepsis, and
tissue biopsies control samples from healthy subjects and/or patients with other diseases, such as chronic liver disease, or sepsis.

Each of the four subsets listed above may be used to train a classification model in its entirety, if present in roughly balanced numbers, or a large population can be subjected to iterative, random undersampling of balanced datasets, in order to achieve a statistically reliable values for coefficients and thresholds for use in a predictive algorithm according to the invention.

Particular embodiments relate to the use of a logistic regression, particularly a ridge regression analysis to obtain a model algorithm which generates a risk score based on the sum of each selected DMR multiplied by an individual weighting value (coefficient). An individual weighting value according to the invention is reflects on the capacity of each DMR to discriminate cancer-containing samples from healthy controls. The risk score may be compared to a threshold value, which accurately separates samples which comprise cancer-derived DNA. The values of the individual weighting values are not particularly limited according to the invention, and depend on the DMR measurements which are chosen for use in a predictive algorithm, the type of classification model used to develop the predictive algorithm, as well as the level of accuracy desired. Examples of such weighting values are presented in Tables 1 to 7.

A threshold according to the invention may be identified by finding the risk score value which discriminates cancer-derive samples, from non-cancer-derived samples with the highest accuracy, for example by finding the value or risk scores with the highest F-score (Sorensen-Dice coefficient, or Dice similarity coefficient). In other words, a threshold applied to the risk scores obtained for a cohort of patients with a known cancer status achieves the highest precision and recall values, wherein a perfect precision and recall is indicated by the value 1. Particular embodiments of the invention relate to a threshold wherein the classification of HCC patients achieves at least a 90%, particularly more than 93%, more particularly more than 95% recall, and at least a 95% precision. Such thresholds appropriate for use in an additive predictive score utilising the methylation values derived from, or applied to specific subsets of DMR according to the invention are demonstrated in Tables 1 through 7.

In particular embodiments of the measurement step according to the invention relating to the use of the methylation level determined for 20 to 38 DMR in an additive linear risk score calculation, the absolute value of the threshold used in the assignment step is between 0.70 to 1.70, particularly between 1.00 to 1.50, more particularly wherein the absolute value of threshold is about 1.23.

Particular embodiments of the assignment step according to the invention relate to a low probability of having cancer which is defined as about a 6% probability of having cancer and/or a high probability of having cancer which is defined as particularly about a 94% probability of having cancer.

Particular embodiments of the invention relate to the use of a patient sample selected from an exploratory biopsy of a tissue in which cancer is suspected to be present, and/or a blood, plasma or serum sample taken from the patient, wherein the DNA is first extracted from the sample, and subsequently treated with a deaminating agent to generate deaminated DNA,

Certain embodiments relate to the use of chemical reagents to selectively modify either the methylated, or unmethylated form of dinucleotide CpG sites present in DNA extracted from the patient sample. The resulting modified CpG may be detected directly, or may be exposed to further reagents which distinguish modified sites. Selective modification of CpG sites may be achieved, for example, using treatment with hydrazine, or bisulphite ions. Hydrazine-treated DNA may be targeted for cleavage by piperidine in order to identify CpG methylation.

Particular embodiments relate to use of bisulphite-treated DNA in a methylation assay, particularly treating DNA from a patient sample with sodium bisulphite. The process converts cytidine residues to uracil, leaving 5-methylcytosine unmodified. Treated DNA may be further contacted with nucleic acid probes designed to hybridize to either a cytosine or uracil present at a certain site in order to distinguish a methylated or non-methylated locus respectively. Probe binding may be assessed by quantitative methodology such as sequencing, quantitative polymerase chain reaction, or a methylation chip array, such as those manufactured by Illumina used to measure DNA methylation levels in the patient sample cohorts analysed in the examples. In embodiments relating to the use of DNA sequencing methodology to obtain a DNA methylation level at a CpG site, methylated cytosines are indicated by the presence of a cytosine, whereas unmethylated residues are read as a thymine residue.

The methylation of a CpG site may be measured by methods sensitive to the methylation status of a CpG dinucleotide known in the art, including, but not limited to next generation sequencing, quantitative polymerase chain reaction, or a methylation array.

Particular embodiments relate to the use of a beta methylation value obtained using a methylation array.

In some embodiments the measurement step comprises contacting deaminated DNA prepared from a patient sample with a nucleic acid probe specific for a certain CpG site.

Particular embodiments related to contacting deaminated DNA prepared from a patient sample with a nucleic acid probe which bears a fluorescent label. Examples include, but are not limited to a TaqMan probe, or the nucleic acid probes of a methylation array.

In some embodiments, the nucleic acid probe specific for one of the specified CpG sites is used in a sequencing reaction in order to determine the level of DNA methylation at the CpG. In particular embodiments, two probes are used to specifically hybridize to, thereby detecting and quantifying, the methylated and unmethylated sequences. In such embodiments, one probe can be employed that is specific for a sequence generated by a conversion reaction, for example effected by an enzyme capable of converting unmethylated cytosines to uracil, or bisulfite conversion, which similarly converts C to U. Another probe is employed to specifically hybridize to the methylated site, which is not affected by conversion. The two probes may be labelled by different fluorescent dyes capable of being detected in the same reaction mix on different fluorescent channels.

For nanopore detection, specific primers are used to amplify the converted or unconverted sequence, respectively, which is then sequenced directly.

Particular embodiments of the method according to any one of the previous embodiments or aspects of the invention relate to a method comprising measuring a DNA methylation level of 8 to 20 of the DMR specified in Table 1 in DNA extracted from a patient sample, wherein one of the DMR is DMR 1, in order to determine whether a hepatocellular carcinoma (HCC) DNA methylation signature is present in a patient sample.

The invention further encompasses the use of one or more nucleic acid probes which bind in a methylation-dependent manner to one or more of the specified CpG sites in each of ≥3, particularly ≥8-10, more particularly ≥20 of the DMR1 to DMR38 as specified above for use in the manufacture of a kit for the detection of condition hepatocellular carcinoma DNA in human tissue samples or cell-free samples including plasma and serum.

In particular embodiments, the kit is provided for regular screening (particularly at annual, more particularly at biannual intervals) of liquid blood samples obtained from a patient diagnosed with cirrhosis, to enable early detection of liver cancer.

In particular embodiments, the method according to the invention is applied to a sample obtained from a patient who has previously been diagnosed with cirrhosis. In some particular embodiments, the sample is obtained from a patient diagnosed with Hepatitis C.

In particular embodiments, the method according to the invention is applied to a sample obtained from a patient previously diagnosed with cirrhosis, in order to determine the likelihood that the patient will go on to develop, or has already progressed to a type of liver cancer, particularly HCC. In more particular embodiments, the method is applied as a regular screening strategy to a patient diagnosed with cirrhosis, for example, in 6 month intervals, in order to determine if the patient has progressed to liver cancer, particularly HCC. In certain embodiments, the patient assigned with a high probability of having cancer, is recommended for more invasive, or costly screening protocols, such as MRI, or a liver biopsy procedure.

An additional aspect of the invention relates to a pharmaceutical composition for use in treating a patient having been assigned a high probability of having cancer by a method as specified above, including a patient previously diagnosed with cirrhosis, the composition comprising an antineoplastic therapeutic agent. If the diagnostic method specified above identifies a patient, such as but not limited to a cirrhosis patient, in which cancer is relatively advanced, particularly wherein imaging and or tumour histopathological analyses are performed subsequent to assignment of a high probability of having cancer, reveal metastasis, such as to organs other than the liver, portal invasion, or a performance status classification of 1 or 2 has been assigned, a chemotherapeutic agent is provided. In certain embodiments, the chemotherapeutic agent is selected from lenvatinib, regorafenib, cabozantinib, ramucirumab, or sorafenib. In particular embodiments, the chemotherapeutic agent is sorafenib. In alternative embodiments, the drug is a checkpoint inhibitor selected from the group of antibodies reactive to a checkpoint regulatory molecule comprised in the group of CTLA-4 (Uniprot P16410), PD-1 (Uniprot Q15116), PD-L1 (Uniprot Q9NZQ7), B7H3 (CD276; Uniprot Q5ZPR3), VISTA (Uniprot Q9H7M9), TIGIT (UniprotQ495A1), TIM-3 (HAVCR2, Uniprot Q8TDQ0), CD158 (killer cell immunoglobulin-like receptor family), TGF-beta (P01137). In particular embodiments, the drug is selected from the group comprised of ipilimumab (Bristol-Myers Squibb; CAS No. 477202-00-9), nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), Avelumab (Merck KGaA; CAS No. 1537032-82-8), Durvalumab (Astra Zenaca, CAS No. 1428935-60-7), and Cemiplimab (Sanofi Aventis; CAS No. 1801342-60-8).

A further aspect of the invention relates to a method of treating a cirrhosis patient having been assigned with a high probability of having cancer according to the method outlined herein, in combination with the outcome of imaging and/or histopathological tumour analysis, in accordance with the recommended clinical application provided by the Barcelona-Clinic Liver Cancer staging system (Khorsandi S. E., HBP Surgery 2012, 2012:154056, the contents of which are incorporated by reference herein in their entirety).

The invention encompasses a method of treating a patient who has been previously diagnosed with cirrhosis wherein the patient has been classified as having a high likelihood of having cancer according to the method as specified in any one of the aspects and embodiments recited above. If the patient is classified as likely to have cancer, as opposed to viral- or alcohol-associated cirrhosis, then the patient is treated according to the clinical best practice of treating liver cancer known to the art, namely in order of application from early, to increasingly late stage intervention:
a resection surgery,
a liver transplantation procedure,
radiofrequency or microwave ablation,
trans-arterial chemoembolization,
a chemotherapeutic agent selected from lenvatinib, regorafenib, cabozantinib, ramucirumab, nivolumab, or pembrolizumab or sorafenib, particularly sorafenib, and/or
immunotherapy by a checkpoint inhibitory agent as disclosed herein, particularly a checkpoint inhibitor selected from the group comprised of ipilimumab (Bristol-Myers Squibb; CAS No. 477202-00-9), nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), Avelumab (Merck KGaA; CAS No. 1537032-82-8), Durvalumab (Astra Zenaca, CAS No. 1428935-60-7), and Cemiplimab (Sanofi Aventis; CAS No. 1801342-60-8).

The described methods provide the ability to provide antineoplastic therapies in only those patients who are most likely to progress from cirrhosis to liver cancer, such as HCC, or cholangiocarcinoma, by first determining if the patient has a high probability of having cancer, as discussed herein, and then treating only those patients so classified.

The method of treating a patient having been previously diagnosed with cirrhosis comprises:
determining in an ex-vivo patient sample, particularly liver biopsy and/or a blood, plasma or serum sample, the methylation level of between 2 to 38, particularly between 8 to 38, more particularly between 8 to 20 differentially methylation regions (DMR) selected from a list comprising or consisting of:
   DMR1 comprising CpG sites (cg)144855744, cg20547777, and/or cg16009311;
   DMR2 comprising cg25366404, cg08864240, cg03422350, cg09655253, and/or cg10791278;
   DMR3 comprising cg07003643, cg10904867, cg16996281, cg19560971, and/or cg09186818;
   DMR4 comprising cg17571559, cg09666573, cg11702866, cg17660833, and/or cg05551003;
   DMR5 comprising cg14021523, cg07040024, and/or cg27088038
   DMR6 comprising cg06753985, cg02457346, and/or cg27146824;
   DMR7 comprising cg16987638, cg22399984, cg09113474, and/or cg04206219;
   DMR8 comprising cg24932457, cg14430141, cg21577836, and/or
   cg09473826;
   DMR9 comprising cg26550936, cg25140531, cg11882607, cg23482898, and/or cg08851782;
   DMR10 comprising cg27528748, cg27108629, and/or cg02475600;
   DMR11 comprising cg20511797, cg13847987, and/or cg13803765;
   DMR12 comprising cg09754845, cg25029797, cg22646311, and/or cg06635328;
   DMR13 comprising cg24224304, cg00512726, cg25936177, cg16179969, cg07726953, cg24569447, and/or cg10151685;
   DMR14 comprising cg10759972, cg02860599, and/or cg08625822;
   DMR15 comprising cg24202448, cg03920764, and/or cg09845293;
   DMR16 comprising cg09816096, cg22151985, and/or cg08901057;
   DMR17 comprising cg23551720, cg24095592, and/or cg03260240;
   DMR18 comprising cg05469574, cg12432526, cg04172640, and/or cg06862949;
   DMR19 comprising vcg26134665, cg02043600, cg03793804, cg25033993, cg07537206, cg03144232, and/or cg05787209;
   DMR20 comprising cg09343092, cg03368099, cg25390165, cg20817131, cg01323381, cg03744763, cg14013695, cg05774699, cg03207666, cg12015737, cg14058329, cg19643053, cg07049592, cg02106682, cg27151303, cg21641458, cg14882265, cg05579037, cg13694927, cg17432857, cg23454797, cg08070327, cg25506432, cg00969405, cg01748892, cg26023912, and/or cg16997642;
   DMR21 comprising cg21591742, cg03918304, cg25371634, cg18115040, cg13217260, cg20649017, and/or cg17489939;
   DMR22 comprises cg26465391, cg08668790, cg01268824, cg21790626, cg05661282, cg12506930, cg03142586, cg11294513, cg27049766, and/or cg03234186;
   DMR23 comprises cg05105207, cg04024865, and/or cg01887388;
   DMR24 comprises cg07003643, cg10904867, cg16996281, cg19560971, and/or cg09186818;
   DMR25 comprising cg08992305, cg00393585, cg12861945, cg06481168, cg11630554, cg25904183 and/or cg20697094;
   DMR26 comprising cg05670004, cg06999856, cg26768075, cg16692735, and/or cg02613809;
   DMR27 comprising cg15699085, cg04071270, and cg06883126;
   DMR28 comprising cg18512232, cg27110938, cg13806267, cg25877512, cg15909725, cg05033439, cg03134809, cg18431486, and/or cg01998856;
   DMR29 comprising cg26882224, cg04886934, and/or cg17057098;
   DMR30 comprising cg07481320, cg14931854, and/or cg24520538;
   DMR31 comprising cg19885761, cg17847520, cg23495748, cg07295964, cg10312572, cg22776578, cg14648916, 0, cg18909295, cg18328894, and/or cg15630459;
   DMR32 comprising cg10237990, cg16800851, cg18411550, cg08358392, cg18798995, cg08106148, cg07826275, cg24516147, and/or cg09710740;
   DMR33 comprising cg11044099, cg12120367, cg00583001, cg26831001, cg04600055, and/or cg17398515;
   DMR34 comprising cg00603340, cg26600753, cg17279652, and/or cg12717963;
   DMR35 comprising cg02532030, cg22136013, cg08313040, cg02375585, cg11715943, cg17664233, cg01309395, cg18927185, cg05547391, cg12208000, and/or cg15737123;
   DMR36 comprising cg15712310, cg01635555, cg01744822, cg06984903, and/or cg01394847;
   DMR37 comprising cg19846168, cg00779565, cg15203905 and/or cg23640231;
   DMR38 comprising cg24428372, cg24737408, cg23900228m cg01144768, and/or cg22405774,
wherein the methylation level of the DMR is the methylation level of one, or the average of 2 or more CpG sites comprised within said DMR to provide a plurality of DMR methylation levels;
   and wherein the methylation level determined for DMR2, DMR4, DMR5, DMR9, DMR10, DMR14, DMR15, DMR16, DMR18, DMR23, DMR24, DMR28, DMR29, DMR35, and/or DMR37 indicates hypermethylation of the DMR, and/or
   the methylation level determined for DMR1, DMR3, DMR6, DMR7, DMR8, DMR11, DMR12, DMR13, DMR17, DMR19, DMR20, DMR21, DMR22, DMR25, DMR26, DMR27, DMR30, DMR31, DMR32, DMR33, DMR34, DMR36, and/or DMR38 indicates hypomethylation of the DMR;
indicating that the patient has hepatocellular carcinoma (HCC), then a treatment selected from the following list is administered to the patient:
   surgical resection, or a liver transplantation procedure;
   radiofrequency ablation or microwave ablation;
   an effective dose of a chemotherapy agent, particularly a chemotherapy agent selected from lenvatinib, regorafenib, cabozantinib, ramucirumab, nivolumab, or pembrolizumab or sorafenib, more particularly sorafenib.

The invention further encompasses the use of primers, and adequate oligonucleotide probes, in addition to quantitative PCR and/or sequencing equipment for use in the manufacture of a kit for the detection of HCC.

The method may be embodied by way of a computer-implemented method, particularly wherein the evaluation and the assignment step are executed by a computer.

Further, the method may be embodied by way of a computer program, comprising computer program code, that when executed on the computer cause the computer to execute at least the evaluation and/or assignment step. Particularly, the results of the measurement step may be provided to the computer and/or the computer program by way of a user input and/or by providing a computer-readable file comprising information regarding the methylation level obtained during the measurement step. Results from the measurement step may be stored for further processing on a memory of the computer, on a non-transitory storage medium.

In an alternative aspect, the invention provides a system for determining the risk or likelihood of a subject having cancer. In particular embodiments, the cancer is lung, colon, breast, or liver cancer. In more particular embodiments, the system determines whether a liver disease patient has developed or is at high risk of recurrence of HCC. In one embodiment, the system comprises a plurality of probes, designed and configured (capable of revealing) to detect (probe or reveal) the level of methylation, i.e. hypermethylation or hypomethylation at differentially methylation regions (DMR) as identified herein. In one particular embodiment, the plurality of probes comprises a set of two probes for each DMR, one capable of specifically hybridizing to the methylated sequence and another capable of specifically hybridizing to the sequence generated from the unmethylated sequence by conversion. The system includes a device designed and configured for reading out the level of each probes' signal as well as a computer (electronic computing device) and a computer program, wherein the computer program comprises computer program code that when executed on the computer causes the computer to perform the methods steps according to any one of aspects of the invention outlines above. For example, calculated a mean methylation value for redundant CpG probes within a DMR, or applying weighted values to the methylation levels of multiple DMR and incorporating them into a patent classification algorithm.

In another embodiment, the system comprises a methylation array, capable of detecting hypermethylation or its absence at differentially methylation regions (DMR) as identified herein.

Wherever alternatives for single separable features such as, for example, different subsets of DMR or different selections of CpG sites within each DMR, or types of cancer are laid out herein as “embodiments”, it is to be understood that such alternatives may be combined freely to form discrete embodiments of the invention disclosed herein. Thus, any of the alternative embodiments for a DMR may be combined with any of the alternative embodiments of a type of cancer and these combinations may be combined with any diagnostic method mentioned herein.

The invention is further illustrated by the following examples and figures, from which further embodiments and advantages can be drawn. These examples are meant to illustrate the invention but not to limit its scope.

### DESCRIPTION OF THE FIGURES

FIG. 1 shows an overview of the DNA methylation datasets assembled. a) number of samples across different types, i.e. HCC tumour, healthy liver and cirrhotic and other liver diseased samples. b) number of samples per study constituting the Train & Test dataset. c) similar to b), number of samples per study constituting the Validation dataset.

FIG. 2 shows optimisation of number of top DNA methylation HCC biomarkers. Greedy sequential DMRs selection selects the best DMR for sequential addition to an LinearSVC model. For each number of DMRs, 30 balanced train sets were generated and benchmarked. Models were trained with balanced train sets and used to predict the train, the test and the validation datasets. The number of features to be selected ranges from 1 to 38, where the latter represents the median number of features in the LinearSVC models. Error margins represent the 95th confidence interval.

FIG. 3 shows HCC biomarker DMR benchmarking analysis. Comparison of the leave-one-out recall and precision rates obtained by the multiple HCC biomarker sets for a) the tissue samples and b) the cfDNA samples. c) precision and recall rates of the multiple HCC biomarker feature sets trained using the Train & Test samples and predicting on the independent Validation set. d) Heat map showing mean beta methylation value of the HCC and non-HCC (healthy, cirrhosis, and chronic liver disease) samples in the Train & Test sample subset.

FIG. 4 shows ranking of HCC DNA methylation risk score features. a) DMR coefficients across the 1,000 permutations of the balanced datasets. b) Left: The precision and recall of the top 1 to 38 DMRs was tested by training on the Train & Test dataset, and testing using the Validation dataset. Right: Ridge classifier DMR coefficients from the Top 38 and Top 20 DMR signatures. Solid black line represents a linear regression and 95% confidence interval. Dashed line represents a diagonal. c) Precision-recall curves of the Validation samples calculated using linear risk score estimated from the mean coefficients obtained in the 1,000 permutation analysis.

FIG. 5 shows DMR signature risk score a) precision-recall curve ranking exclusively samples in the Train & Test dataset that were not used to identify and estimate the HCC biomarkers and weights. Maximum F1-score along the curve is represented with and “x” and the DMR signature risk score threshold at the given recall and precision. Random precision is drawn as a dashed horizontal line. b) DMR signature risk score Train & Test samples not used for HCC biomarker discovery plotted against a representative top performing DMR. Vertical line represents the DMR signature risk score threshold found at the maximum F1-score in a) and the associated recall and precision rates are reported. c) precision-recall curve of all cfDNA samples of the Train & Test dataset including samples from patients with other types of cancer (labelled as “Cancer”). d) similar to b), DMR signature risk score threshold, vertical dashed line, is estimated from the maximum F1-score point along the precision-recall curve in c) and recall and precision rates are reported. e) DMR signature risk score estimated for the Validation set samples plotted against two highly predictive HCC DMRs and their methylation profiles. DMR signature risk score threshold defined using the Train & Test dataset. Precision and recall rates reported are those estimated in the Validation dataset.

FIG. 6 shows benchmarking and performance metrics DMR signature risk score. a) DMR signature risk score calculated for all the samples in the Train & test dataset which were not used for the identification of the DMR signature risk score biomarker DMR values and their weights. DMR signature risk score plotted against three top predictive HCC DNA methylation biomarkers. HCC classification threshold is represented by a dashed vertical line and precision and recall rates are reported. b) As in a) only cfDNA samples are utilised and cfDNA samples from patients with other cancers (marked as blue and labelled as “Cancer”) are also considered as a positive event. cfDNA samples from healthy controls are marked in green (“Healthy”)- Recall and precision rates are reported.

FIG. 7 shows how the mean and standard error a) recall and b) precision of the DMR signature risk score model is altered by random undersampling of only 1, 2 or 3 CpG sites within each DMR and estimated their mean methylation using only these CpG sites for the top 8, 10, 20 or 38 DMRs.

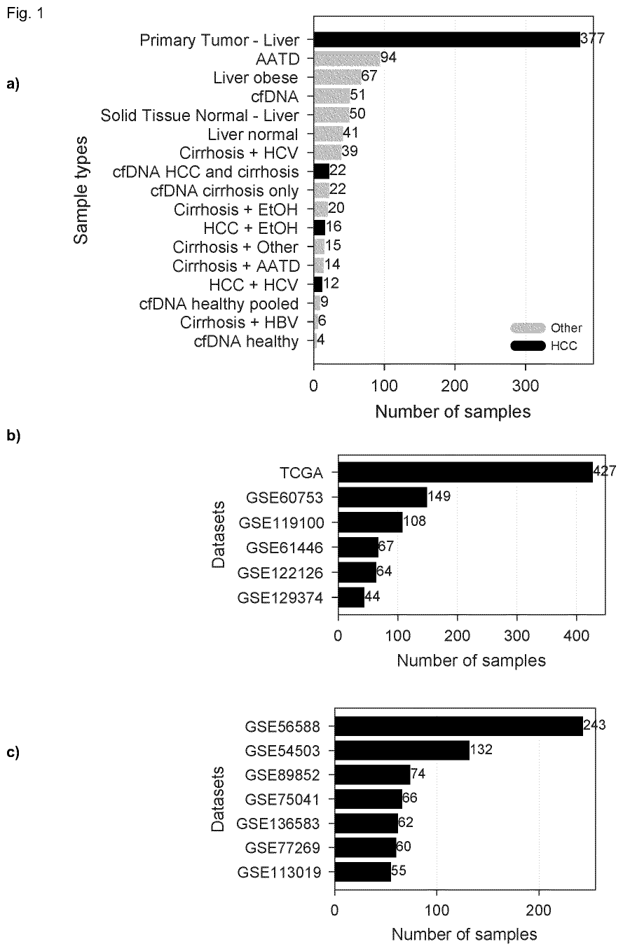

Table 1. shows the 38 predictive differentially methylated regions (DMR), the mean is the weighting value (coefficient) identified using iterative ridge regression analysis, the DMR signature risk score threshold and performance recall and precision calculated using data from all 38 DMR to classify samples in the Test & Train data set. Also shown, Cluster annotation used for bioinformatic DMR identification, the genomic location of the DMR on human reference genome 38 (hg38), the CpG sites measured by microarray probes evaluated within each DMR, and the relative average methylation of each DMR in the HCC samples, compared to the non HCC samples in the Train & Test data set.

Table 2. shows the mean weighting value (coefficient) identified for a selection of 20 DMR using a linear regression classifier ridge regression analysis as in Table 1, standard deviation (StD), and the DMR signature risk score threshold and performance calculated for recall and precision.

### EXAMPLES

### Example 1

### DNA Methylation Dataset for the Discovery of HCC Biomarkers

To define a comprehensive set of DNA methylation biomarkers for the detection of HCC from tissue and plasma cfDNA samples HCC-related studies characterising genome-wide DNA methylation changes were identified, using high-throughput Illumina-based, Infinium 450K and EPIC assays. A train and test set matching the criteria defined above 859 samples was assembled from 6 different studies covering: HCC tissue and cfDNA samples from HCC patients; cirrhotic tissue from multiple aetiologies, and cfDNA from cirrhotic patients; healthy liver tissue; and other non-HCC diseased tissue (e.g. liver obesity and Alpha 1 antitrypsin deficiency), and cfDNA from non-HCC patients (e.g. sepsis and other cancer types).

A level of DNA methylation was available for total of 452,567 methylation sites (CpG sites) are measured and methylation levels represented using beta methylation values, ranging between 0, hypomethylated, and 1, hypermethylated. All datasets were merged into a single matrix containing signal intensities imported from the raw IDAT files and processed using the functional normalisation pipeline (Fortin, J. P et al. 2014, Genome Biol. 15: 503). The ratio between the methylation and unmethylation channels was calculated and exported as beta methylation values (β) [EQ1] with an offset of 100 (the recommended standard offset for Illumina methylation arrays) and rounded to 5 decimal places:

$$β = \frac{Methylated}{Methylated + Unmethylated + offset} \qquad [ EQ 1 ]$$

For the downstream analyses several filtering steps were taken: (i) probes containing a single nucleotide polymorphism (SNP) in the CpG site or in the single nucleotide extension at a minor allele frequency (MAF) lower than 0.01 were excluded from downstream analysis; (ii) using maxprobes R package (v0.0.2) cross-reactive probes of the Illumina methylation arrays were removed; (iii) CpG sites with missing values were discarded; (iv) an updated probe annotation mapped to the hg38 reference build and probes with no available alignments were not considered; and (v) to focus on biomarkers that are gender agnostic CpG sites mapping to sex chromosomes X and Y were removed from downstream analyses. The final filtered DNA methylation matrix covered a total of 390,445 CpG sites without any missing value across all samples.

Additionally, a Validation dataset containing 692 tissue samples was assembled from 7 independent datasets in which original data or publication was not accessible but processed beta methylation values was available. This validation dataset comprises multiple studies with distinct experimental and analytical pipelines as independent validation of the approaches used in this study. Overall, the assembled >1,500 whole-genome DNA methylation arrays represents an heterogenous and comprehensive resource to discover and validate DNA methylation biomarkers of HCC clinically relevant diseased backgrounds, such as cirrhosis.

### Unsupervised Selection of High Quality and Informative DNA Methylation Regions

HCC patient samples showed distinct patterns of multiple and clustered CpG sites with hypo and hyper methylation profiles. CpG clusters were defined as spanning at least 3 CpG sites, such that two consecutive sites are at most 500 base-pairs (bp) apart using the clusterMaker function from Bump Hunter R package (v1.30.0). The CpG clusters were overlapped with the filtered CpG sites defined as above, and only CpG clusters with at least 3 CpG sites with measurements were considered. A final CpG cluster matrix was defined by taking the mean of all filtered CpG sites within each cluster region, generating a DNA methylation matrix spanning 39,868 CpG clusters, to reduce the impact of potential confounder effects, and to focus on genomic regions, instead of individual CpG sites, to reveal robust and generalisable biomarkers for HCC.

### Discovery of Methylation Regions Predictive of HCC

To differentiate HCC from a background of cirrhotic samples in tissue and cfDNA, a linear support vector machine classifiers (LinearSVC) was trained to find methylation regions predictive of HCC, using a leave-one-out cross validation strategy, where one sample at a time was left out for testing the prediction, while the other 858 samples were used as a training set.

Differentially methylated and predictive regions were identified by using balanced datasets in a two-step approach. Firstly, differentially methylated regions (DMR) are identified by removing potential cofounder effects, i.e. sex, age, global methylation and tumour purity. A differential methylation analysis between HCC (HCC-T and HCC-CF) and cirrhotic (C-T and C-CF) samples was then performed, incorporating the previous variables as covariates in a linear modelling order to account for their potential impact. Only significantly differentially methylated CpG clusters (likelihood-ratio test FDR<1%) were selected for model training,

DMRs are defined as those CpG clusters with a ratio test and ANOVA FDR lower than 1%. Thus, a median of 1,355 DMRs across the leave-one-out procedure. A total of 88 samples, containing equal proportions of HCC and cirrhotic, and cfDNA and tissue samples, were used for model training. In order to retain a maximum of information relevant to the smaller set of cfDNA samples, when analysing a cfDNA sample, this sample was not used for training and the total number of samples in each class is therefore reduced to 21, hence a total of 84 evenly distributed samples are used instead.

### Evaluation, Comparison and Assembly of HCC Methylation Signature

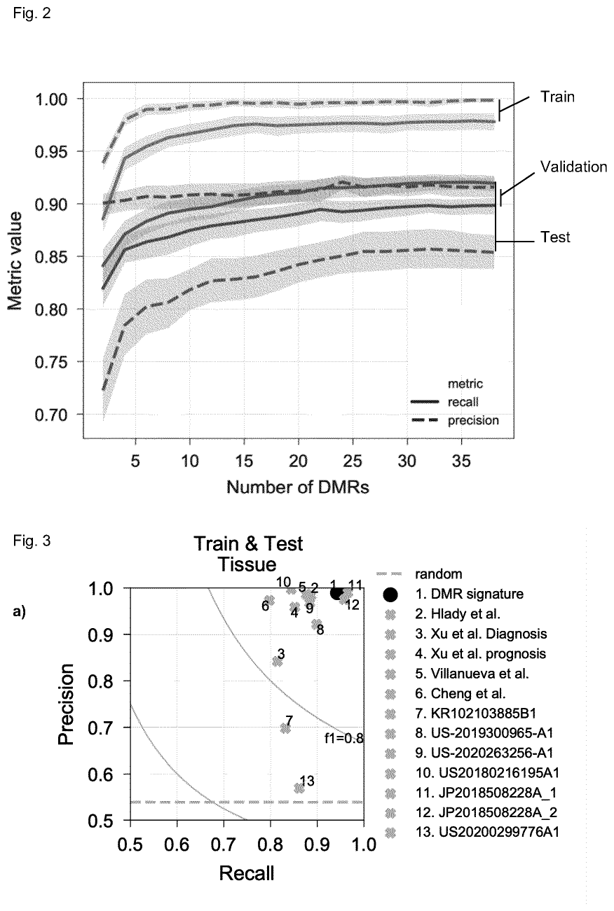

To find the optimal number of DMRs for a predictive methylation signature, the addition of DMRs into a feature set was sequentially tested, while assessing the precision and recall of the resulting LinearSVC models. The DMRs identified in HCC samples were assessed for their ability to predict HCC by training linear support vector machines (LinearSVC) using a L1 regularization, with penalty parameter (C) set to 1.5, to reduce the number of DMRs considered in the model. DMRs with non-zero weights in the trained model are then defined as the most predictive DMRs to classify HCC samples. A median of 38 HCC predictive DMRs were identified per model across the 859 iterations of the leave-one-out procedure, where 150 unique DMRs are found in at least 5% of all trained models (n=43). Recall and precision increased steeply up to 10 DMRs, and from that point the test and validation datasets show small but consistent increments in performance. Together with the fact that frequency of each DMRs in the optimal models is positively correlated with its absolute mean effect size, the top 38 most frequent DMRs in the leave-one-out cross validation procedure (FIG. 2).

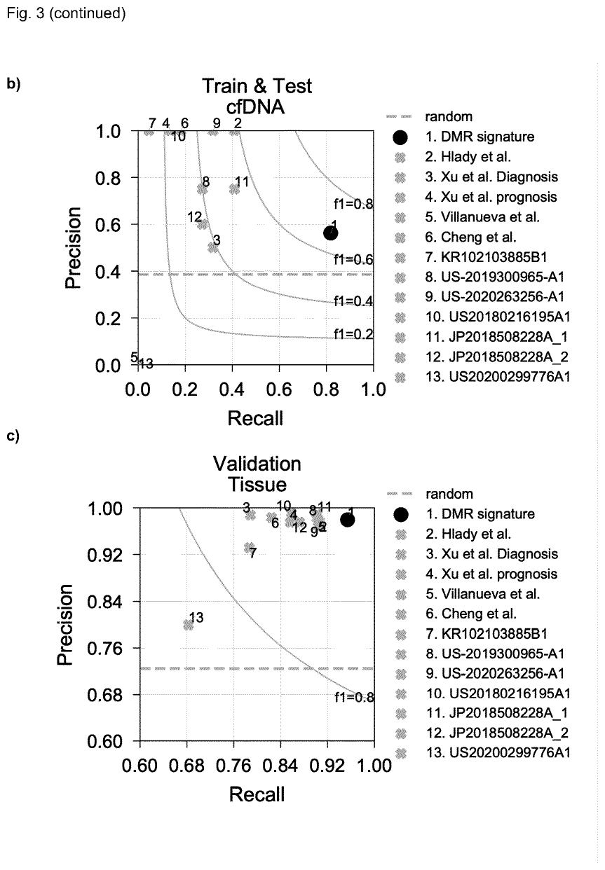

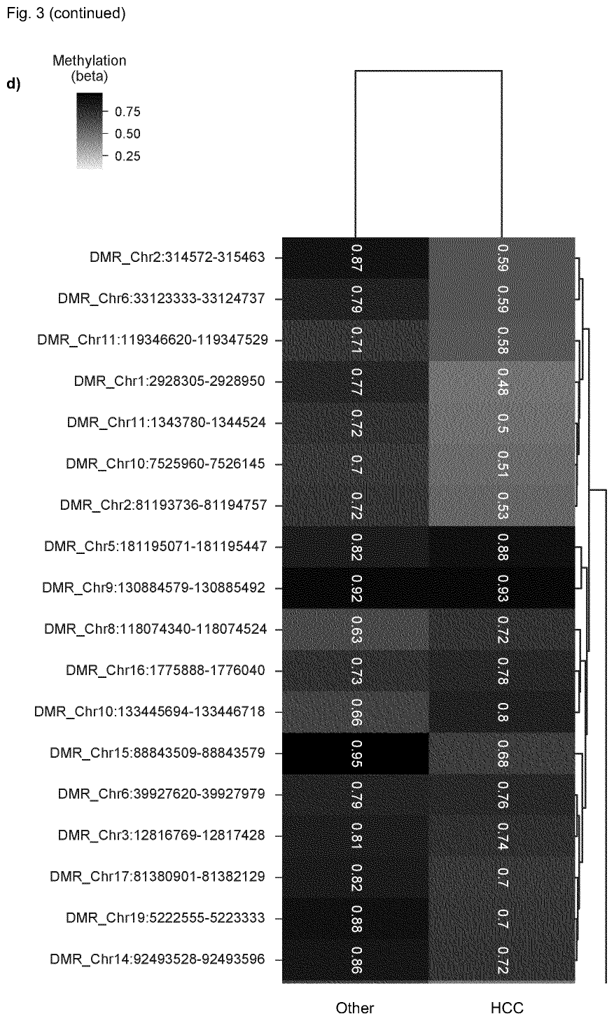

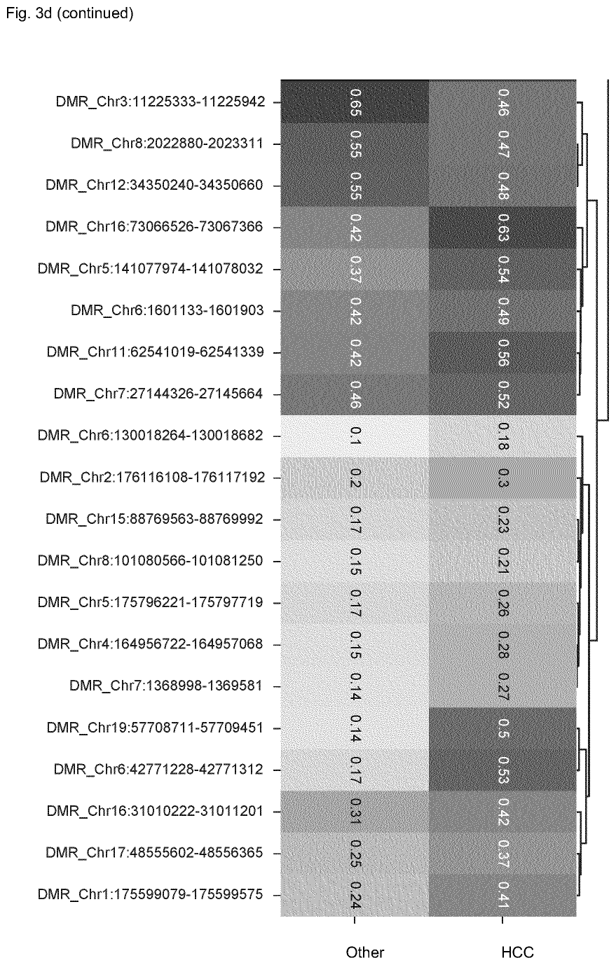

The resulting DNA methylation signature was compared against other similar approaches assembled from the literature, comprising 13 sets of CpG sites from 4 publications (Villanueva A. et al. 2015. Hepatology 61:1945; Hlady R. A. et al. 2019. Theranostics 9:7239; Cheng J. et al. 2018. Genome Med. 10:42; Aran D. et al. 2015. Nat. Commun. 6:8971) and 7 patents (KR102103885B1; US2019300965A1; US20180216195A1; US2020263256A1; JP2018508228A1; JP2018508228A2; US20200299776A1). To avoid potential methodological bias, an ensemble model using logistic and linear classification models was used, in contrast to the support vector machine model used previously. This approach iteratively predicts the HCC status of the sample that is left-out for testing in a leave-one-out cross validation. The performance of all models was estimated using multiple standard performance metrics, i.e., recall, precision, accuracy, Mathew's correlation coefficient (MCC) and balanced accuracy Overall precision and recall scores for tissue samples were greater than 0.8 (FIG. 3a) and all models had a poorer performance when predicting the subset of cfDNA samples while precisions were less affected (FIGS. 3a and 3b). The Validation tissue samples dataset was then used as an independent benchmark, and it was observed that overall feature sets provided a mean precision of 96% and recall rates 86% (FIG. 3c). The signature of this study obtained the highest recall (95%) while preserving precision (98%) (FIG. 3c).

This approach confirms a signature of hyper and hypo methylated regions can successfully distinguish HCC samples from cirrhotic, healthy and other non-HCC samples, and benchmarks positively against other DNA methylation signatures, particularly showing low false negative rates, i.e. high recall, both in tissue and cfDNA samples.

### Methylation Diagnostic Metric for Hepatocellular Carcinoma

The top 38 DMRs, encompassing a total of 214 CpG sites out of which 118 and 74 showed significant hyper and hypo methylation in HCC (FIG. 3d, Table 1), were then used to define a single metric that could encompass the information from a whole DNA methylation signature to use as a diagnostic metric for early detection of HCC.

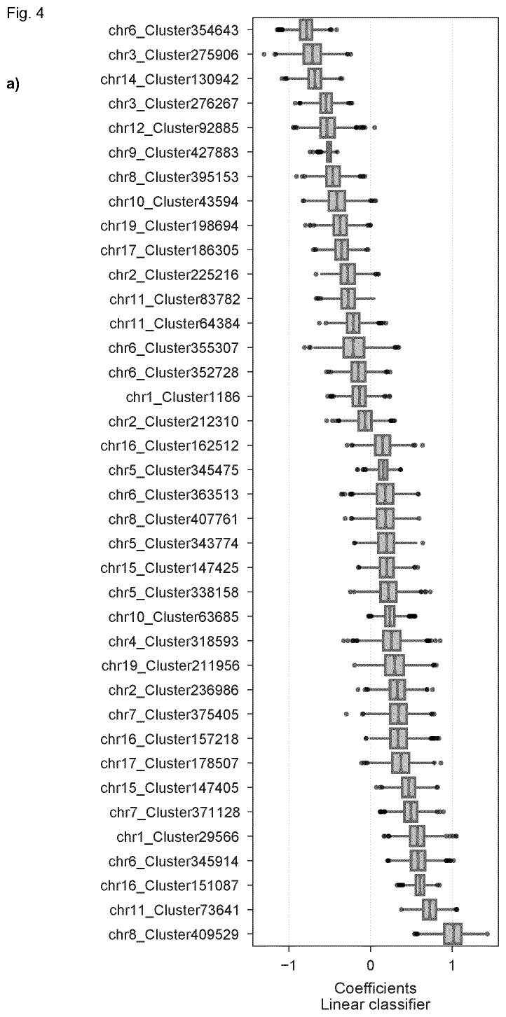

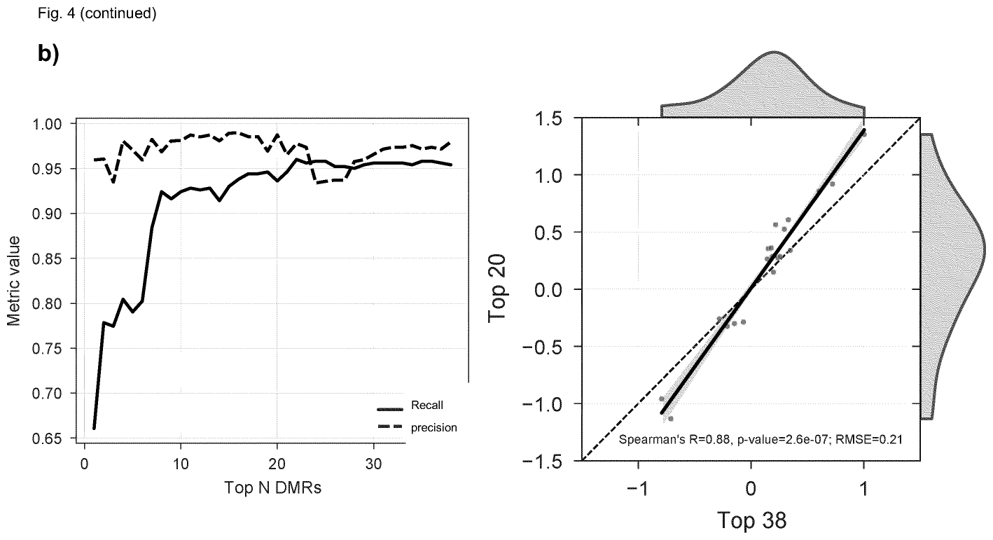

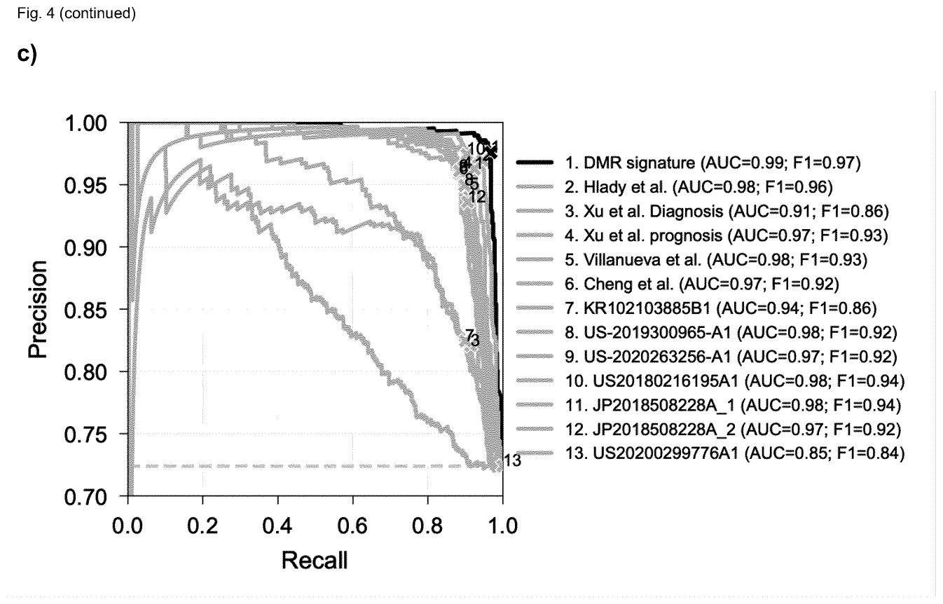

The importance and variability of each DMR in the signature was estimated by randomly generating 1,000 balanced training datasets as above, and training a linear regression classifier (FIG. 4a). Secondly, an additive linear score (DMR signature risk score) was developed, consisting of the sum of each 38 DMR of the methylation signatures, weighted by their signed mean coefficients learnt by each model. In other words, DMRs with high absolute mean coefficients across all trained models were given higher preponderance in the score. In brief, the linear risk score is an integrated score of the top 38 DMRs recurrently present with non-zero weights in the linear support vector machines (LinearSVCs) trained with the balanced sample sets in the leave-one-out cross validation. The preponderance (weight) of each DMR was estimated using 1,000 permutations of a balanced dataset used to train a Ridge classifier with an alpha parameter set to 1, ensuring a regularisation of the model's feature coefficients (individual weighting values), while preserving them as non-zero. The mean and standard deviation of each DMR is then calculated across all 1,000 iterations. The mean coefficients are then used in a weighted additive score where features with larger absolute scores have larger preponderance in the linear DMR signature risk score. Based on this feature set and weights a score is calculated for each sample. Recall and precision curves were generated using the risk score and the HCC status of the samples. Optimal threshold and precision and recall rates are estimated based on the best F1 metric possible along the curves.

The top 38 DMRs were arranged in descending order of importance (absolute mean coefficient, Table 1) and the precision and recall of the top 1 to 38 DMRs was tested by training on the Train & Test dataset, and testing using the Validation dataset. Here too, precision remained relatively stable, while recall increased steeply up to 8 to 10 DMRs, from 10 to 22 the test and validation datasets show small but consistent increments in performance, and from 22-38 marginal improvement can be inferred from the gradual stabilisation of assessed metrics (FIG. 4b). Coefficients are estimated according to the chosen subset of DMR by fitting a ridge classifier with a regularization parameter alpha set to 1. This was performed independently for each Top 2, 3, 5, 8, 10, 20 and 38 signatures resulting in adjusted coefficients and thresholds for HCC classification (Table 1 and 2, FIG. 4b). The selected subsets of DMRs could accurately classify HCC patients from healthy or cirrhotic controls, as demonstrated by the recall and precision values. Models were trained using a random under-sampling of tissue biopsy HCC and cirrhotic samples together with a same number of HCC and cirrhotic liquid biopsy samples, ensuring similar representation of each class. The random undersampling is performed 1,000 times for each Top 20 and Top 38 signatures.

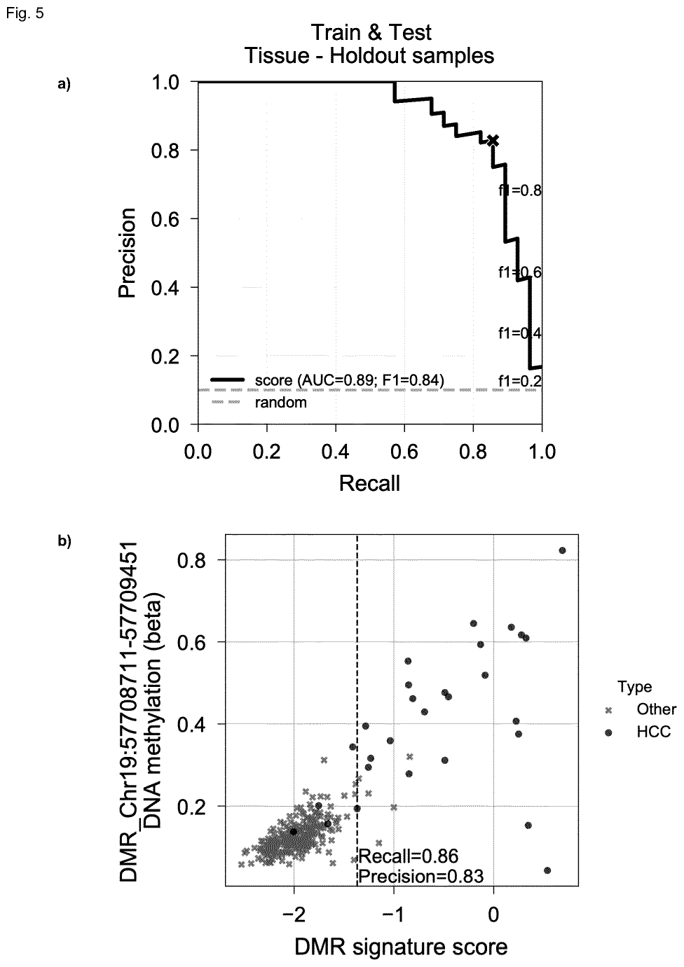

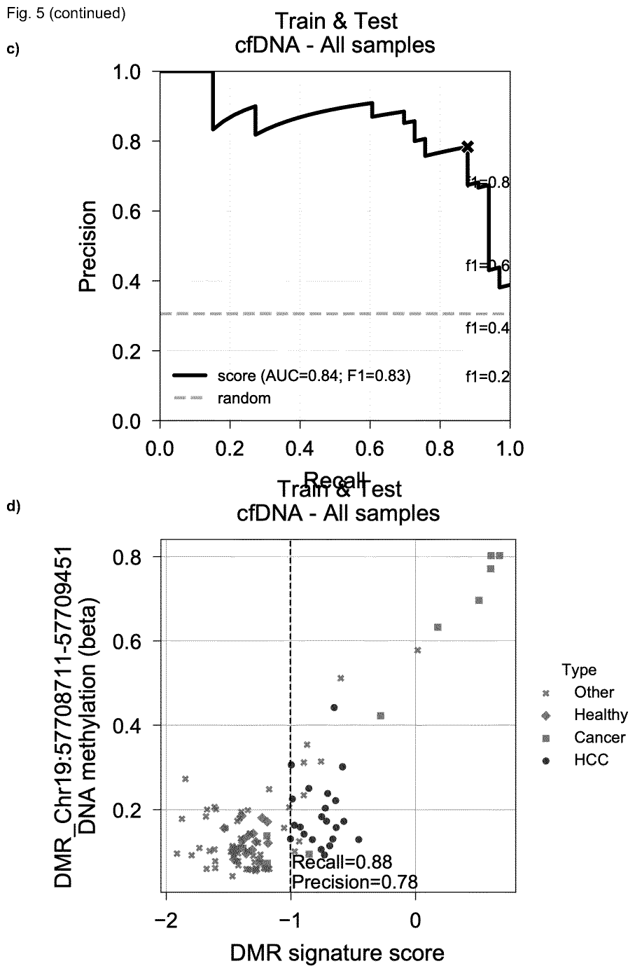

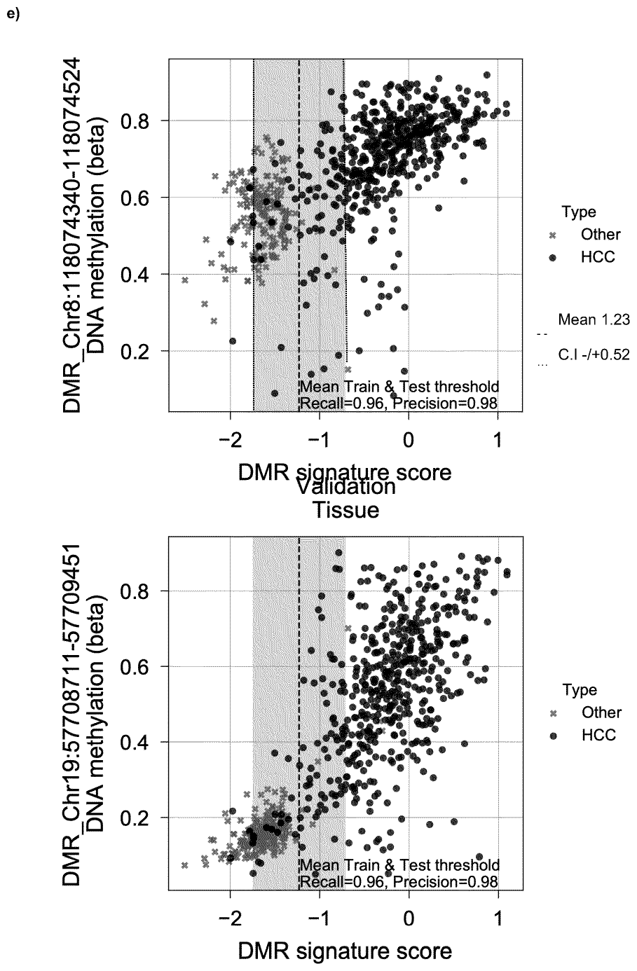

A DMR signature risk score was calculated for all samples in the Test and Train and Validation dataset, and samples were ranked according to probably assignment to HCC. A linear risk score was estimated for other CpG site signatures, and it was observed that in the independent Validation dataset the score based on the DMRs signature outperformed and provided very accurate predictions of HCC (FIG. 4c). Furthermore, in samples from the Train & Test dataset that were held out from the training of the DMR signature and score, the DMR signature risk score clearly split the HCC from non-HCC samples, with a recall (sensitivity) of 86% and precision of 83% (FIG. 5a and b).

CfDNA samples have noisier backgrounds in terms of methylation signals due to the low proportion of DNA derived from tumour compared to a tumour biopsy sample, but are relevant for early-stage diagnostic approaches due to the ease of acquiring liquid samples such as plasma or blood in comparison to tissue biopsies. In addition to the HCC and cirrhotic cfDNA samples, cfDNA samples from healthy controls, sepsis, and patients with cancers from other tissues, including lung, breast and colon were also assessed. Here too, the HCC metric clearly separated the cfDNA HCC and cirrhotic samples used for training of the signature and score.

The DMR signature risk score trained using mainly HCC cancer samples could also perfectly identify cfDNA samples from patients with other cancers compared to sepsis or healthy controls (recall=88% and precision=78%) (FIG. 5c and d). This suggests that HCC biomarkers detect differentiated methylation across multiple cancers. Inclusively, the risk score derived from the top 38 DMR successfully classified HCC samples and identified 7 cfDNA samples (out of 11) from other malignancies, including breast, lung and colorectal cancer

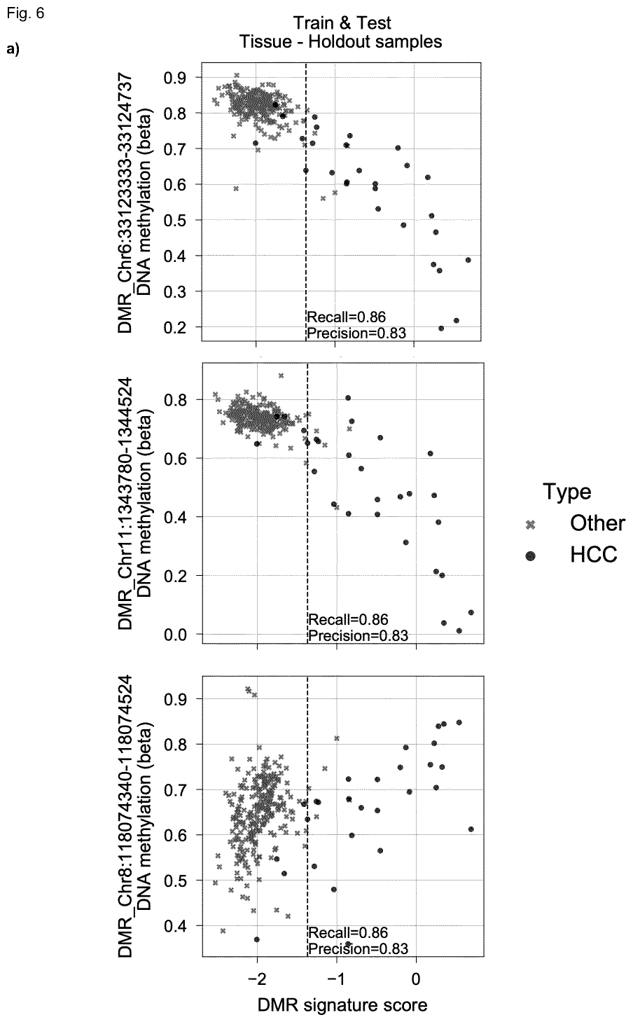

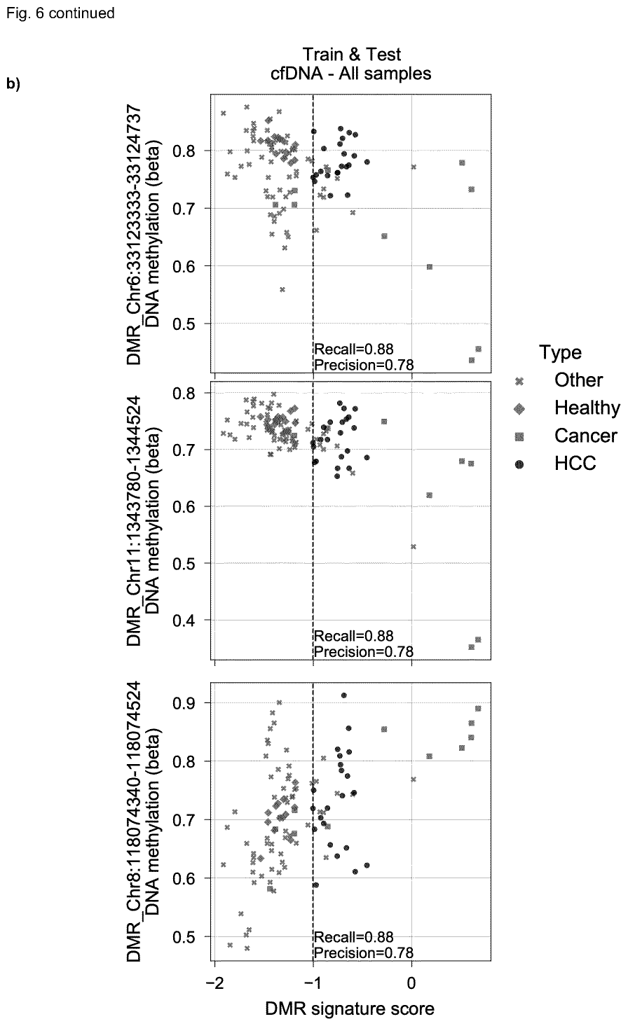

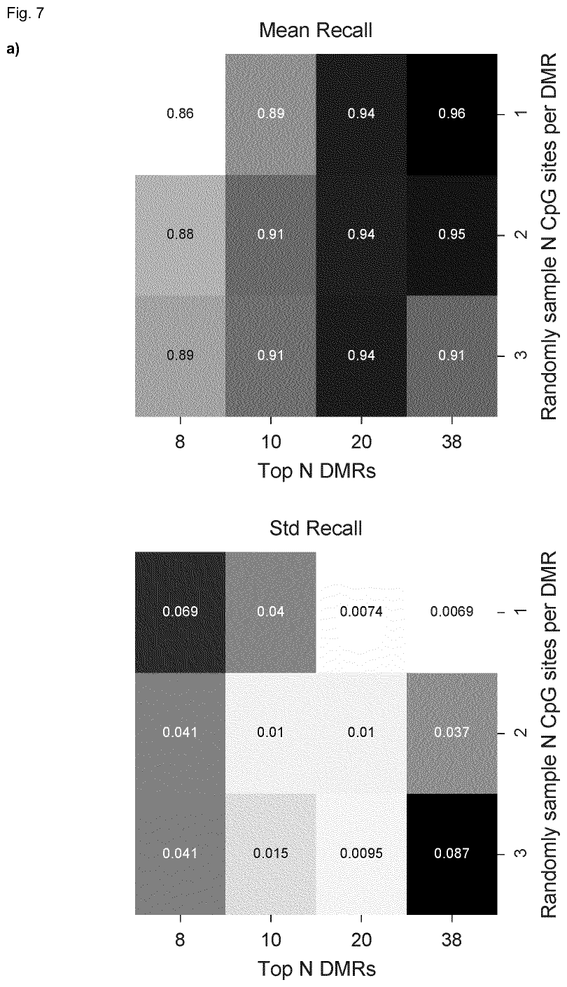

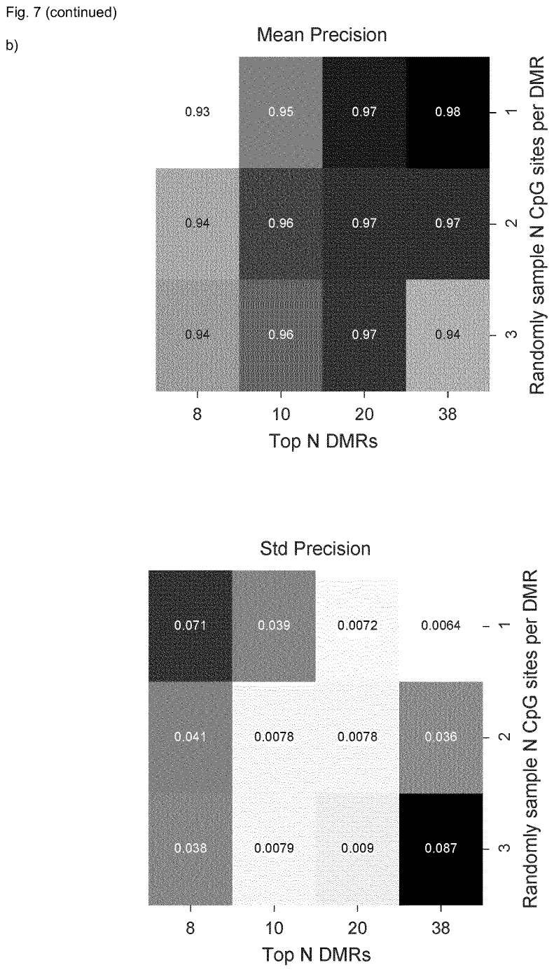

The linear risk score is a valuable metric for the diagnosis of HCC with robust predictive power across many different datasets (FIG. 5e) with heterogeneous backgrounds, and most importantly both in tissue and liquid biopsies (FIG. 6). The redundancy of the multiple CpG sites identified in each DMR was confirmed by performing a random undersampling of either 1, 2, or 3 CpG sites to contribute towards the methylation level of the top 8, 10, 20 or 38 DMR. Recall was observed to increase with the number of Top DMRs used, independently of the number of CpG sites considered per DMR (FIG. 7).

The DMR signature risk score provided incorporates information from differential methylation regions (DMRs) which encompass multiple consecutive CpG sites with similar methylation profiles, providing robust biomarkers for liquid biopsies, and compares favourably against multiple DNA methylation signatures of HCC from publications and patents.

TABLE 1

| | | | | | | % methylated |
|---|---|---|---|---|---|---|
| DMR | Cluster | mean | StD | DMR hg38 position | CG sites | HCC |
| DMR1 | chr8_Cluster409529 | 1 | 0.16 | DMR_Chr8: 118074340-118074524 | cg144855744, cg 20547777, cg16009311 | 88 |
| DMR2 | chr6_Cluster354643 | −0.79 | 0.11 | DMR_Chr6: 39927620-39927979 | cg25366404, cg08864240, cg03422350, | 104 |
| | | | | | cg09655253, cg10791278 | |
| DMR3 | chr11_Cluster73641 | 0.72 | 0.12 | DMR_Chr11: 62541019-62541339 | cg07003643, cg10904867, cg16996281, | 75 |
| | | | | | cg19560971, cg09186818 | |
| DMR4 | chr3_Cluster275906 | −0.71 | 0.15 | DMR_Chr3: 11225333-11225942 | cg17571559, cg09666573, cg11702866, | 141 |
| | | | | | cg17660833, cg05551003 | |
| DMR5 | chr14_Cluster130942 | −0.68 | 0.12 | DMR_Chr14: 92493528-92493596 | cg14021523, cg07040024, cg27088038 | 119 |
| DMR6 | chr16_Cluster151087 | 0.6 | 0.08 | DMR_Chr16: 1775888-1776040 | cg06753985, cg02457346, cg27146824 | 94 |
| DMR7 | chr6_Cluster345914 | 0.59 | 0.13 | DMR_Chr6: 1601133-1601903 | cg16987638, cg22399984, cg09113474, | 86 |
| | | | | | cg04206219 | |
| DMR8 | chr1_Cluster29566 | 0.57 | 0.13 | DMR_Chr1: 175599079-175599575 | cg24932457, cg14430141, cg21577836, | 59 |
| | | | | | cg09473826 | |
| DMR9 | chr3_Cluster276267 | −0.55 | 0.1 | DMR_Chr3: 12816769-12817428 | cg26550936, cg25140531, cg11882607, | 109 |
| | | | | | cg23482898, cg08851782 | |
| DMR10 | chr12_Cluster92885 | −0.53 | 0.14 | DMR_Chr12: 34350240-34350660 | cg27528748, cg27108629, cg02475600 | 115 |
| DMR11 | chr9_Cluster427883 | −0.51 | 0.04 | DMR_Chr9: 130884579-130885492 | cg20511797, cg13847987, cg13803765 | 99 |
| DMR12 | chr7_Cluster371128 | 0.49 | 0.12 | DMR_Chr7: 1368998-1369581 | cg09754845, cg25029797, cg22646311, | 52 |
| | | | | | cg06635328 | |
| DMR13 | chr15_Cluster147405 | 0.47 | 0.11 | DMR_Chr15: 88769563-88769992 | cg24224304, cg00512726, cg25936177 | 74 |
| | | | | | cg16179969, cg07726953, cg24569447 | |
| | | | | | cg10151685 | |
| DMR14 | chr8_Cluster395153 | −0.46 | 0.12 | DMR_Chr8: 2022880-2023311 | cg10759972, cg02860599, cg08625822 | 117 |
| DMR15 | chr10_Cluster43594 | −0.41 | 0.15 | DMR_Chr10: 7525960-7526145 | cg24202448, cg03920764, cg09845293 | 137 |
| DMR16 | chr19_Cluster198694 | −0.37 | 0.12 | DMR_Chr19: 5222555-5223333 | cg09816096, cg22151985, cg08901057 | 126 |
| DMR17 | chr17_Cluster178507 | 0.37 | 0.15 | DMR_Chr17: 48555602-48556365 | cg23551720, cg24095592, cg03260240 | 68 |
| DMR18 | chr17_Cluster186305 | −0.35 | 0.11 | DMR_Chr17: 81380901-81382129 | cg05469574, cg12432526, cg04172640 | 117 |
| | | | | | cg06862949 | |
| DMR19 | chr16_Cluster157218 | 0.35 | 0.15 | DMR_Chr16: 31010222-31011201 | cg26134665, cg02043600, cg03793804 | 74 |
| | | | | | cg25033993, cg07537206, cg03144232 | |
| | | | | | cg05787209 | |
| DMR20 | chr7_Cluster375405 | 0.34 | 0.15 | DMR_Chr7: 27144326-27145664 | cg09343092, cg03368099, cg25390165, | 88 |
| | | | | | cg20817131, cg01323381, cg03744763, | |
| | | | | | cg14013695, cg05774699, cg03207666, | |
| | | | | | cg12015737, cg14058329, cg19643053, | |
| | | | | | cg07049592, cg02106682, cg27151303, | |
| | | | | | cg21641458, cg14882265, cg05579037, | |
| | | | | | cg13694927, cg17432857, cg23454797 | |
| | | | | | cg08070327, cg25506432, cg00969405 | |
| | | | | | cg01748892, cg26023912, cg16997642 | |
| DMR21 | chr2_Cluster236986 | 0.33 | 0.13 | DMR_Chr2: 176116108-176117192 | cg21591742, cg03918304, cg25371634 | 67 |
| | | | | | cg18115040, cg13217260, cg20649017 | |
| | | | | | cg17489939 | |
| DMR22 | chr19_Cluster211956 | 0.3 | 0.17 | DMR_Chr19: 57708711-57709451 | cg26465391, cg08668790, cg01268824, | 28 |
| | | | | | cg21790626, cg05661282, cg12506930, | |
| | | | | | cg03142586, cg11294513, cg27049766, | |
| | | | | | cg03234186 | |
| DMR23 | chr2_Cluster225216 | −0.28 | 0.12 | DMR_Chr2: 81193736-81194757 | cg05105207, cg04024865, cg01887388 | 136 |
| DMR24 | chr11_Cluster83782 | −0.28 | 0.12 | DMR_Chr11: 119346620-119347529 | cg07003643, cg10904867, cg16996281, | 122 |
| | | | | | cg19560971, cg09186818 | |
| DMR25 | chr4_Cluster318593 | 0.26 | 0.16 | DMR_Chr4: 164956722-164957068 | cg08992305, cg00393585, cg12861945, | 54 |
| | | | | | cg06481168, cg11630554, cg25904183 | |
| | | | | | cg20697094 | |
| DMR26 | chr10_Cluster63685 | 0.24 | 0.09 | DMR_Chr10: 133445694-133446718 | cg05670004, cg06999856, cg26768075, | 83 |
| | | | | | cg16692735, cg02613809 | |
| DMR27 | chr5_Cluster338158 | 0.22 | 0.15 | DMR_Chr5: 141077974-141078032 | cg15699085, cg04071270, cg06883126 | 69 |
| DMR28 | chr11_Cluster64384 | −0.21 | 0.12 | DMR_Chr11: 1343780-1344524 | cg18512232, cg27110938, cg13806267, | 144 |
| | | | | | cg25877512, cg15909725, cg05033439, | |
| | | | | | cg03134809, cg18431486, cg01998856 | |
| DMR29 | chr15_Cluster147425 | 0.2 | 0.12 | DMR_Chr15: 88843509-88843579 | cg26882224, cg04886934, cg17057098 | 140 |
| DMR30 | chr6_Cluster355307 | −0.2 | 0.18 | DMR_Chr6: 42771228-42771312 | cg07481320, cg14931854, cg24520538 | 32 |
| DMR31 | chr5_Cluster343774 | 0.19 | 0.13 | DMR_Chr5: 175796221-175797719 | cg19885761, cg17847520, cg23495748, | 65 |
| | | | | | cg07295964, cg10312572, cg22776578, | |
| | | | | | cg14648916, cg05958740, cg18909295, | |
| | | | | | cg18328894, cg15630459 | |
| DMR32 | chr6_Cluster363513 | 0.18 | 0.15 | DMR_Chr6: 130018264-130018682 | cg10237990, cg16800851, cg18411550, | 56 |
| | | | | | cg08358392, cg18798995, cg08106148, | |
| | | | | | cg07826275, cg24516147, cg09710740 | |
| DMR33 | chr8_Cluster407761 | 0.18 | 0.15 | DMR_Chr8: 101080566-101081250 | cg11044099, cg12120367, cg00583001, | 71 |
| | | | | | cg26831001, cg04600055, cg17398515 | |
| DMR34 | chr5_Cluster345475 | 0.15 | 0.08 | DMR_Chr5: 181195071-181195447 | cg00603340, cg26600753, cg17279652, | 93 |
| | | | | | cg12717963 | |
| DMR35 | chr6_Cluster352728 | −0.15 | 0.12 | DMR_Chr6: 33123333-33124737 | cg02532030, cg22136013, cg08313040, | 134 |
| | | | | | cg02375585, cg11715943, cg17664233, | |
| | | | | | cg01309395, cg18927185, cg05547391, | |
| | | | | | cg12208000, cg15737123 | |
| DMR36 | chr16_Cluster162512 | 0.15 | 0.13 | DMR_Chr16: 73066526-73067366 | cg15712310, cg01635555, cg01744822, | 67 |
| | | | | | cg06984903, cg01394847 | |
| DMR37 | chr1_Cluster1186 | −0.14 | 0.12 | DMR_Chr1: 2928305-2928950 | cg19846168, cg00779565, cg15203905 | 160 |
| | | | | | cg23640231 | |
| DMR38 | chr2_Cluster212310 | −0.07 | 0.12 | DMR_Chr2: 314572-315463 | cg24428372, cg24737408, cg23900228 | 68 |
| | | | | | cg01144768, cg22405774 | |
| | Risk score threshold | −1.23 | 0.52 | | | |
| | recall | 0.93 | 0.11 | | | |
| | precision | 0.97 | 0.12 | | | |

TABLE 2

| DMR | mean | StD |
|---|---|---|
| DMR1 | 1.35 | 0.19 |
| DMR4 | −1.13 | 0.16 |
| DMR2 | −0.96 | 0.11 |
| DMR3 | 0.92 | 0.13 |
| DMR6 | 0.86 | 0.10 |
| DMR21 | 0.61 | 0.15 |
| DMR27 | 0.56 | 0.15 |
| DMR22 | 0.52 | 0.17 |
| DMR33 | 0.36 | 0.16 |
| DMR34 | 0.35 | 0.07 |
| DMR19 | 0.34 | 0.17 |
| DMR28 | −0.32 | 0.13 |
| DMR35 | −0.30 | 0.14 |
| DMR38 | −0.29 | 0.13 |
| DMR31 | 0.29 | 0.15 |
| DMR25 | 0.29 | 0.19 |
| DMR26 | 0.27 | 0.10 |
| DMR36 | 0.26 | 0.15 |
| DMR23 | −0.26 | 0.12 |
| DMR29 | 0.15 | 0.14 |
| threshold | 1.23 | 0.45 |
| recall | 0.95 | 0.01 |
| precision | 0.97 | 0.01 |

TABLE 3

| DMR | mean | StD |
|---|---|---|
| DMR1 | 1.77 | 0.24 |
| DMR4 | −1.10 | 0.17 |
| DMR27 | 1.08 | 0.20 |
| DMR6 | 1.00 | 0.12 |
| DMR2 | −0.96 | 0.12 |
| DMR16 | 0.73 | 0.19 |
| DMR35 | −0.61 | 0.16 |
| DMR31 | 0.60 | 0.15 |
| DMR28 | −0.44 | 0.17 |
| DMR23 | −0.33 | 0.13 |
| threshold | 0.46 | 0.39 |
| recall | 0.91 | 0.01 |
| precision | 0.96 | 0.01 |

TABLE 4

| DMR | mean | std |
|---|---|---|
| DMR1 | 1.91 | 0.25 |
| DMR4 | −1.17 | 0.18 |
| DMR6 | 0.97 | 0.22 |
| DMR7 | −0.97 | 0.14 |
| DMR31 | 0.81 | 0.18 |
| DMR35 | −0.65 | 0.19 |
| DMR28 | −0.37 | 0.19 |
| DMR23 | −0.32 | 0.15 |
| threshold | −0.54 | 0.41 |
| recall | 0.90 | 0.03 |
| precision | 0.95 | 0.03 |

TABLE 5

| DMR | mean | std |
|---|---|---|
| DMR1 | 1.98 | 0.27 |
| DMR4 | −1.18 | 0.18 |
| DMR36 | 1.14 | 0.23 |
| DMR35 | −0.80 | 0.20 |
| DMR28 | −0.54 | 0.21 |
| threshold | 0.18 | 0.38 |
| recall | 0.89 | 0.01 |
| precision | 0.94 | 0.01 |

TABLE 6

| DMR | mean | std |
|---|---|---|
| DMR1 | 2.312 | 0.316 |
| DMR4 | −1.281 | 0.224 |
| DMR28 | −1.222 | 0.214 |
| threshold | −0.073 | 0.415 |
| recall | 0.823 | 0.022 |
| precision | 0.922 | 0.014 |

TABLE 7

| DMR | mean | std |
|---|---|---|
| DMR1 | 2.523 | 0.340 |
| DMR4 | −1.749 | 0.259 |
| threshold | 0.629 | 0.381 |
| recall | 0.822 | 0.019 |
| precision | 0.908 | 0.009 |

1. A method of determining whether a patient has cancer, particularly lung, colon, breast, or liver cancer, more particularly hepatocellular carcinoma, the method comprising:
a. in a measurement step, determining the methylation level of between 2 to 38, particularly between 8 to 38, more particularly between 8 to 20 differentially methylation regions (DMR) in an ex-vivo patient sample, particularly an exploratory biopsy of a tissue in which cancer is suspected to be present, and/or a blood, plasma or serum sample taken from the patient,
wherein the DMR are selected from a list comprising or consisting of:
DMR1 comprising CpG sites (cg) 144855744, cg20547777, and/or cg16009311;
DMR2 comprising cg25366404, cg08864240, cg03422350, cg09655253, and/or cg10791278;
DMR3 comprising cg07003643, cg10904867, cg16996281, cg19560971, and/or cg09186818;
DMR4 comprising cg17571559, cg09666573, cg11702866, cg17660833, and/or cg05551003;
DMR5 comprising cg14021523, cg07040024, and/or cg27088038
DMR6 comprising cg06753985, cg02457346, and/or cg27146824;
DMR7 comprising cg16987638, cg22399984, cg09113474, and/or cg04206219;
DMR8 comprising cg24932457, cg14430141, cg21577836, and/or
cg09473826;
DMR9 comprising cg26550936, cg25140531, cg11882607, cg23482898, and/or cg08851782;
DMR10 comprising cg27528748, cg27108629, and/or cg02475600;
DMR11 comprising cg20511797, cg13847987, and/or cg13803765;
DMR12 comprising cg09754845, cg25029797, cg22646311, and/or cg06635328;
DMR13 comprising cg24224304, cg00512726, cg25936177, cg16179969, cg07726953, cg24569447, and/or cg10151685;
DMR14 comprising cg10759972, cg02860599, and/or cg08625822;
DMR15 comprising cg24202448, cg03920764, and/or cg09845293;
DMR16 comprising cg09816096, cg22151985, and/or cg08901057;
DMR17 comprising cg23551720, cg24095592, and/or cg03260240;
DMR18 comprising cg05469574, cg12432526, cg04172640, and/or cg06862949;
DMR19 comprising vcg26134665, cg02043600, cg03793804, cg25033993, cg07537206, cg03144232, and/or cg05787209;
DMR20 comprising cg09343092, cg03368099, cg25390165, cg20817131, cg01323381, cg03744763, cg14013695, cg05774699, cg03207666, cg12015737, cg14058329, cg19643053, cg07049592, cg02106682, cg27151303, cg21641458, cg14882265, cg05579037, cg13694927, cg17432857, cg23454797, cg08070327, cg25506432, cg00969405, cg01748892, cg26023912, and/or cg16997642;
DMR21 comprising cg21591742, cg03918304, cg25371634, cg18115040, cg13217260, cg20649017, and/or cg17489939;
DMR22 comprises cg26465391, cg08668790, cg01268824, cg21790626, cg05661282, cg12506930, cg03142586, cg11294513, cg27049766, and/or cg03234186;
DMR23 comprises cg05105207, cg04024865, and/or cg01887388;
DMR24 comprises cg07003643, cg10904867, cg16996281, cg19560971, and/or cg09186818;
DMR25 comprising cg08992305, cg00393585, cg12861945, cg06481168, cg11630554, cg25904183 and/or cg20697094;
DMR26 comprising cg05670004, cg06999856, cg26768075, cg16692735, and/or cg02613809;
DMR27 comprising cg15699085, cg04071270, and cg06883126;
DMR28 comprising cg18512232, cg27110938, cg13806267, cg25877512, cg15909725, cg05033439, cg03134809, cg18431486, and/or cg01998856;
DMR29 comprising cg26882224, cg04886934, and/or cg17057098;
DMR30 comprising cg07481320, cg14931854, and/or cg24520538;
DMR31 comprising cg19885761, cg17847520, cg23495748, cg07295964, cg10312572, cg22776578, cg14648916, cg05958740, cg18909295, cg18328894, and/or cg15630459;
DMR32 comprising cg10237990, cg16800851, cg18411550, cg08358392, cg18798995, cg08106148, cg07826275, cg24516147, and/or cg09710740;
DMR33 comprising cg11044099, cg12120367, cg00583001, cg26831001, cg04600055, and/or cg17398515;
DMR34 comprising cg00603340, cg26600753, cg17279652, and/or cg12717963;
DMR35 comprising cg02532030, cg22136013, cg08313040, cg02375585, cg11715943, cg17664233, cg01309395, cg18927185, cg05547391, cg12208000, and/or cg15737123;
DMR36 comprising cg15712310, cg01635555, cg01744822, cg06984903, and/or cg01394847;
DMR37 comprising cg19846168, cg00779565, cg 15203905 and/or cg23640231;
DMR38 comprising cg24428372, cg24737408, cg23900228m cg01144768, and/or cg22405774, and wherein the methylation level of the DMR is the methylation level of one, or the average of 2 or more CpG sites comprised within said DMR to provide a plurality of DMR methylation levels;
b. in an evaluation step, establishing the combined statistical significance of the plurality of DMR methylation levels determined in the measurement step a.,
c. in an assignment step, assigning the patient a high probability of having cancer, or a low probability of having cancer based on the combined statistical significance of the plurality of DMR methylation levels.

2. The method according to claim 1, wherein the patient is assigned a high probability of having cancer
wherein the methylation level determined for DMR2, DMR4, DMR5, DMR9, DMR10, DMR14, DMR15, DMR16, DMR18, DMR23, DMR24, DMR28, DMR29, DMR35, and/or DMR37 indicates hypermethylation of the DMR;
and/or
wherein the methylation level determined for DMR1, DMR3, DMR6, DMR7, DMR8, DMR11, DMR12, DMR13, DMR17, DMR19, DMR20, DMR21, DMR22, DMR25, DMR26, DMR27, DMR30, DMR31, DMR32, DMR33, DMR34, DMR36, and/or DMR38 indicates hypomethylation of the DMR;
and wherein hypermethylation is characterized as a methylation level above an average of methylation level of said DMR as determined in a plurality of control samples previously determined to be free of cancer cells, and wherein hypomethylation is characterized as a methylation level below the average of methylation level of said DMR.

3. The method according to claim 1, wherein in the evaluation step, the plurality of DNA methylation levels is submitted to a predictive algorithm which classifies the sample as according to the probability that the sample contains DNA derived from a cancer cell to obtain a risk score, particularly wherein the algorithm is an additive linear score, more particularly wherein the plurality of DNA methylation levels is submitted to an additive linear score by
multiplying each of the plurality of DMR methylation levels by an individual weighting value according to a relative predictive power of each DMR to obtain a plurality of weighted DMR methylation values, and
calculating the sum of the plurality weighted DMR methylation values to obtain a risk score.

4. The method according to claim 3, wherein in the assignment step the risk score is compared to a threshold,
wherein a risk score equal or above (≥) a threshold indicates that the patient has a high probability of having cancer,
and wherein a risk score below (<) the threshold indicates that the patient has a low probability of having cancer,
particularly wherein in the measurement step, the methylation level of 20 to 38 DMR is determined, and wherein in the assignment step the absolute value of the threshold is between 0.70 to 1.70, particularly between1.00 to 1.50, more particularly wherein the absolute value of threshold is about 1.23.

5. The method according to claim 1, wherein in the measurement step, the plurality of DMR for which a DMR methylation level is determined comprises DMR1,
particularly DMR1 and DMR4,
more particularly DMR1, DMR4, and DMR28,
even more particularly DMR1, DMR4, DMR28, DMR35, and DMR36,
still more particularly DMR1, DMR4, DMR6, DMR7, DMR31, DMR35, DMR28 and DMR23,
still more particularly DMR1, DMR4, DM27, DMR6, DMR2, DMR16, DMR31, DMR35, DMR28 and DMR23.

6. The method according to claim 4, wherein the predictive algorithm is obtained by training a classification model, particularly a logistic classification model, or elastic net classification model, more particularly a ridge regression classification model,
and wherein the classification model is trained using a plurality of methylation values obtained from of a plurality of patient samples with a known cancer status comprising equal numbers of
ii. a plurality of cancer patient tissue samples, particularly HCC patient samples, and
iii. a plurality of control samples, particularly a combination of chronic liver disease patient samples and healthy control samples,
iv. wherein the plurality of cancer patient tissue samples, and the plurality of control samples each comprise an equal number of tissue biopsy samples, and cell-free liquid biopsy samples, respectively.

7. The method according to claim 1, wherein in the assignment step
a low probability of having cancer is defined as about a 6% probability of having cancer and/or
a high probability of having cancer is defined as particularly about a 94% probability of having cancer.

8. A method according to claim 1,
comprising obtaining a patient sample selected from an exploratory biopsy of a tissue in which cancer is suspected to be present, and/or a blood, plasma or serum sample taken from the patient, and
extracting DNA from the sample, and
treating the extracted DNA with a deaminating agent to generate deaminated DNA.

9. The method according to claim 1, wherein in the methylation value for a given CpG site is determined using a method selected from next generation sequencing, quantitative polymerase chain reaction, or a methylation array, particularly wherein the methylation value is a beta methylation value obtained using a methylation array.

10. The method according to claim 1, wherein the patient sample is a plasma sample.

11. The method according to claim 1, wherein the patient sample is an exploratory biopsy sample of tissue suspected of having cancer.

12. The method according to claim 1, wherein the cancer is hepatocellular carcinoma (HCC).

13. A pharmaceutical composition for use in treating a patient previously diagnosed with cirrhosis, the composition comprising:
an antineoplastic drug selected from lenvatinib, regorafenib, cabozantinib, ramucirumab, or sorafenib, particularly sorafenib; and/or
a checkpoint inhibitor, particularly a checkpoint inhibitor selected from the group comprised of ipilimumab, nivolumab, pembrolizumab, pidilizumab, atezolizumab, avelumab, durvalumab, and cemiplimab, more particularly of nivolumab, or pembrolizumab;
wherein the patient has been assigned a high probability of having cancer by a method as specified in claim 1.

14. A system for determining whether a patient has cancer, particularly lung, colon, breast, or liver cancer, more particularly hepatocellular carcinoma, the system comprising:
a set of probes designed and configured to reveal a methylation level of a DMR;
a device designed and configured to read a probes' signal; and
a computer as well as a computer program, wherein the computer program comprises computer program code that when executed on the computer causes the computer to perform the methods steps according to claim 1.
